(19) United States
(12) Patent Application Publication
(10) Pub. No.: US 2024/0300900 A1
(43) Pub. Date: Sep. 12, 2024

(54) HYDROPHILIC AZADIBENZOCYCLOOCTYNE DERIVATIVES AND METAL-FREE CLICK REACTIONS WITH THESE HYDROPHILIC AZADIBENZOCYCLOOCTYNE DERIVATIVES

(71) Applicant: Roche Diagnostics Operations, Inc., Indianapolis, IN (US)

(72) Inventors: Frank Bergmann, Iffeldorf (DE); Dieter Heindl, Muenchen (DE); Nils Winter, München (DE)

(21) Appl. No.: 18/660,802

(22) Filed: May 10, 2024

Related U.S. Application Data
(63) Continuation of application No. PCT/EP2022/081339, filed on Nov. 9, 2022.

(30) Foreign Application Priority Data
Nov. 10, 2021 (EP) 21207445.4

(51) Int. Cl. C07D 225/08; A61K 47/54; A61K 49/00; A61K 51/04

(52) U.S. Cl. CPC C07D 225/08; A61K 47/545; A61K 49/0052; A61K 51/044

(57) ABSTRACT

The invention relates in a first aspect to an azadibenzocyclooctyne derivative according to formula (I) or a salt thereof having specific substituents at the benzo rings of the DIBAC structure and having specific substituents connected to the nitrogen atom of the DIBAC structure. A second aspect of the invention is directed to a conjugate of formula (II), wherein a substituent R⁶ is connected to the N atom of the 8 membered ring of the DIBAC structure via a linker structure —C(═O)-[L]ₙ-Z—. A third aspect of the invention relates to a method for the modification of a target molecule, wherein a conjugate according to the second aspect is reacted with a target molecule comprising a 1,3-dipole group or a 1,3-(hetero)diene group. In a fourth aspect, the invention is directed to the use of the conjugate according to the second aspect for bioorthogonal labeling and/or modification of a target molecule. A fifth aspect of the invention relates to a modified target molecule comprising the reaction product of a conjugate according to the second aspect and a target molecule comprising a 1,3-dipole group or a 1,3-(hetero)diene group, obtained or obtainable from the method of the third aspect. In a sixth aspect, the invention is related to a kit comprising a modified target molecule according to the fifth aspect as detector reagent and a suitable capture reagent.

The invention relates in a first aspect to an azadibenzocyclooctyne derivative according to formula (I) or a salt thereof having specific substituents at the benzo rings of the DIBAC structure and having specific substituents connected to the nitrogen atom of the DIBAC structure. A second aspect of the invention is directed to a conjugate of formula (II), wherein a substituent R⁶ is connected to the N atom of the 8 membered ring of the DIBAC structure via a linker structure —C(═O)-[L]n-Z—. A third aspect of the invention relates to a method for the modification of a target molecule, wherein a conjugate according to the second aspect is reacted with a target molecule comprising a 1,3-dipole group or a 1,3-(hetero)diene group. In a fourth aspect, the invention is directed to the use of the conjugate according to the second aspect for bioorthogonal labeling and/or modification of a target molecule. A fifth aspect of the invention relates to a modified target molecule comprising the reaction product of a conjugate according to the second aspect and a target molecule comprising a 1,3-dipole group or a 1,3-(hetero)diene group, obtained or obtainable from the method of the third aspect. In a sixth aspect, the invention is related to a kit comprising a modified target molecule according to the fifth aspect as detector reagent and a suitable capture reagent.

### RELATED ART

Bioorthogonal reactions are widely used in modern chemical biology for the modification of biomolecules. Herein, the [3+2] cycloaddition of an azide with an alkyne leading to a stable 1,2,3-triazole bas an outstanding position as it is most frequently used, i.e. the so called strain-promoted azide-alkyne cycloaddition (SPAAC). An alternative approach is the so called strain-promoted alkyne-nitrone cycloaddition (SPANC). Even though these reactions commonly require copper catalysis, there are variants which are taking advantage of the inherent ring strain (strain promoted azide alkyne cycloaddition, SPAAC) of e.g. cyclic octynes and therefore circumventing the need of any catalyst.

WO 2014/189370 A1 discloses substituted dibenzoazacyclooctyne (DIBAC) derivatives, which carry specific substituents at the benzo rings. Debets et al. (Chem. Commun. 2010, 46, 97-99) also describes DIBAC derivatives which carry specific substituents at the N atom of the 8 membered ring of the DIBAC structure. A synthetic route for preparing DIBAC analogues is also disclosed by Debets et al. (Org. Biomol. Chem., 2014, 12, 5031-5037).

Nevertheless, the known DIBAC derivatives are usually hydrophobic molecules and lead to conjugates that suffer from poor solubility in aqueous solutions and are vulnerable to undesired hydrophobic interactions. So far, no sufficiently water-soluble derivatives have been reported.

Thus, the technical problem underlying the invention was the need for DIBAC derivatives with an improved solubility in aqueous solutions and a minimum of hydrophobic interactions.

### SUMMARY OF THE INVENTION

The problem is solved by the invention with the features of the independent patent claims. Advantageous developments of the invention, which can be realized individually or in combination, are presented in the dependent claims and/or in the following specification and detailed embodiments.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once, typically will be used only once when introducing the respective feature or element. In the following, in most cases, when referring to the respective feature or element, the expressions “at least one” or “one or more” will not be repeated, notwithstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding alternative embodiments of the invention, without any restrictions regarding the scope of the invention and without any restriction regarding the possibility of combining the features introduced in such a way with other optional or non-optional features of the invention.

### 1^{st} ASPECT—AZADIBENZOCYCLOOCTYNE DERIVATIVE

In a first aspect, the invention relates to an azadibenzocyclooctyne derivative according to formula (I) or a salt thereof,

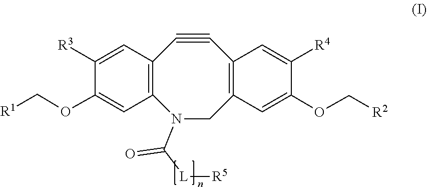

wherein
R¹, R² are independently selected from the group consisting of
[(CH₂)ₐCR^{x}R^{y}]_{b}R^{z} group, wherein a is either zero or an integer from the range of from 1 to 4, b is either zero or an integer from the range of from 1 to 3, R^{x}, R^{y}, R^{z} are independently selected from the group consisting of hydrogen atom, C1 to C3 alkyl group and (CH₂)_{c}SO₃⁻ group, with c being either zero or an integer from the range of from 1 to 4, wherein at least one of R^{x}, R^{y}, R^{z} is a (CH₂)_{c}SO₃⁻ group with the condition:
   if R^{z} is a (CH₂)_{c}SO₃⁻ group with c being zero, then R^{x}, R^{y} are not both a (CH₂)_{c}SO₃⁻ group wherein c is zero, or
   if a is zero, then R^{x} and R^{y} are not both a (CH₂)_{c}SO₃⁻ group wherein c is zero;
      and
—[CR^{r}R^{s}]_{d}—R^{t} group, wherein d is an integer selected from the range of from 1 to 10, R^{r} is selected from the group consisting of hydrogen atom, hydroxyl group and —[CR′(OH)]e—H group, R^{s} is either a hydrogen atom or a —[CR″(OH)]_{f}—H group, and R^{t} is selected from the group consisting of hydrogen atom, C1 to C5 alkyl group and —[CR″′(OH)]_{g}—H group, wherein each R′, R″, R″′ is independently either a hydrogen atom or a —[CH(OH)]ₕ—H group, and each of d, e, f, g and h is independently an integer selected from the range of from 1 to 10, with the condition that if R^{t} is a hydrogen atom or a C1 to C5 alkyl group, then at least one of R^{r}, R^{s} is not a hydrogen atom;
   R³, R⁴ are independently selected from the group consisting of hydrogen atom, C1-C3-alkyl group, halogen atom and —O—C1-C3-alkyl group; and
   R⁵ is selected from the group consisting of carboxyl group, activated carboxyl group and —NHR^{5a} group, wherein R^{5a} is a hydrogen atom or a C1-C5 alkyl group;
   L comprises a chain of covalently bonded atoms forming a backbone and having a length in the range of from 1 to 100 atoms (linker); and
   n is either zero or 1 if R⁵ is a carboxyl group or an activated carboxyl group or n is 1 if R⁵ is a —NHR^{5a} group.

If the index “n” of the linker L is zero, then the linker L is absent and the C atom of the C═O and R⁵ are directly connected by a single bond. The —[CR^{r}R^{s}]_{d}—R^{t} group indicated for R¹, R² represents a straight or branched poly hydroxyl structure. The —[(CH₂)ₐCR^{x}R^{y}]_{b}R^{z} group indicated for R¹, R² represents a residue of at least one sulfonic acid group.

The azadibenzocyclooctyne derivative according to formula (I) or a salt thereof are significantly improved with respect to solubility in aqueous solutions and have therefore a broad suitability to be used in aqueous systems. Already the azadibenzocyclooctyne derivatives according to formula (I) or salts thereof carrying —[CR^{r}R^{s}]_{d}—R^{t} groups have an improved hydrophilicity which is still further improved in the compounds of formula (I) having [(CH₂)ₐCR^{x}R_{y}]_{b}R^{z} groups. Furthermore, the azadibenzocyclooctyne derivatives according to formula (I) or salts thereof allow to avoid unwanted hydrophobic interactions like protein aggregation and therefore potential nonspecific binding in diagnostic assays. The described azadibenzocyclooctyne derivatives according to formula (I) or salts thereof circumvent the need of copper catalysis for the cycloaddition when, optionally carrying further subsitutents as in the conjugates of formula (II) described herein below in more detail, reacted with a target molecule comprising a 1,3-dipole group or a 1,3-(hetero)diene group.

The linker L as comprised in formula (I) comprises, preferably consists of, a chain of atoms forming a backbone, wherein the backbone has a length in the range of from 1 to 100 atoms, preferably a length in the range of from 4 to 50 atoms, more preferably a length in the range of from 5 to 20 atoms, more preferably a length in the range of from 6 to 15 atoms. All atoms forming the backbone are covalently bonded to each other. In one embodiment, the backbone consists of carbon atoms and one or more heteroatoms selected from O, N and S, optionally comprising at least one aryl, heteroaryl, substituted aryl or substituted heteroaryl group (wherein e.g. a phenylene ring accounts for a length of four atoms). Heteroatoms at interior positions are unsubstituted or have one or more substituents selected from the group consisting of hydrogen atom, C1-C5 alkyl and ═O. In some embodiments, the one or more heteroatom(s) are part of a linkage, wherein the linkage is preferably selected from the group consisting of amide linkage, ester linkage, ether linkage, carbamate linkage and urea linkage. Carbon atoms in the chain of atoms of the backbone are substituted with one or more substituents selected from the group consisting of hydrogen atom and C1 to C10 alkyl group. The term “alkyl” by itself or as part of another substituent, means, unless otherwise stated, a straight or branched chain, or cyclic hydrocarbon radical, or combination thereof, having the number of carbon atoms designated (i.e. C1-C10 means one to ten carbon atoms). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, and the like. In one embodiment, the backbone consists of two or more straight alkyl chain segments with one or more heteratom(s) in between the segments. Preferably, the backbone has a length in the range of from 6 to 15 atoms and consists of two or more straight alkyl chain segments, which are preferably unsubstituted, with one or more heteratom(s) in between the segments, wherein the one or more heteroatoms are selected from O and N. More preferably, the backbone has a length in the range of from 6 to 10 atoms and consists of two straight alkyl chain segments, which are preferably unsubstituted, with a linkage between the segments being selected from the group consisting of an ether linkage, an urea linkage, a carbamate linkage and an amide linkage.

### Salts of Azadibenzocyclooctyne Derivative

In cases where R¹ and/or R² comprise —SO₃⁻ group(s), said group(s) is/are present in deprotonated form, wherein the negative charge is preferably compensated by a suitable cation, which is preferably selected from alkali metal cation, preferably Na⁺ or K⁺, and trialkylammonium cation NR^{k}R^{p}R^{q}, wherein preferably R^{k}, R^{p}, R^{q} are independently a C1-C6 alkyl group, more preferably R^{k}, R^{p}, R^{q} are identical and are each a C1-C6 alkyl group. If R⁵ is a carboxyl group, said group is either present in its protonated form or in its deprotonated form, wherein the negative charge is preferably compensated by a suitable cation, which is preferably selected from alkali metal cation, preferably Na⁺ or K⁺, and trialkylammonium cation NR^{k}R^{p}R^{q}, wherein preferably R^{k}, R^{p}, R^{q} are independently a C1-C6 alkyl group, more preferably R^{k}, R^{p}, R^{q} are identical and are each a C1-C6 alkyl group. A preferred trialkylammonium cation is N,N,N-triethylammonium.

According to an embodiment of the azadibenzocyclooctyne derivative or salt thereof, R³, R⁴ are independently a hydrogen atom or a methyl group, preferably R³, R⁴ are identical and are each a hydrogen atom.

According to an embodiment of the azadibenzocyclooctyne derivative or salt thereof,
R¹, R² are each a —[(CH₂)ₐCR^{x}R^{y}]_{b}R^{z} group, wherein for each R¹, R² independently, a is either zero or an integer from the range of from 1 to 4, b is either zero or an integer from the range of from 1 to 3, R^{x}, R^{y}, R^{z} are selected from the group consisting of hydrogen atom, C1 to C3 alkyl group and (CH₂)_{c}SO₃⁻ group, with c being either zero or an integer from the range of from 1 to 4, wherein at least one of R^{x}, R^{y}, R^{z} is a (CH₂)_{c}SO₃⁻ group with the condition:
   if R^{z} is a (CH₂)_{c}SO₃⁻ group with c being zero, then R^{x}, R^{y} are not both a (CH₂)_{c}SO₃⁻ group wherein c is zero, or
   if a is zero, then R^{x} and R^{y} are not both a (CH₂)_{c}SO₃⁻ group wherein c is zero.

According to an embodiment of the azadibenzocyclooctyne derivative or salt thereof,
R¹, R² are each a —[(CH₂)ₐCR^{x}R^{y}]_{b}R^{z} group, wherein for each R¹, R² independently, a is either zero or an integer from the range of from 1 to 4, b is either zero or an integer from the range of from 1 to 3, R^{x} and R^{z} are selected from the group consisting of hydrogen atom, C1 to C3 alkyl group and (CH₂)_{c}SO₃⁻ group with c being either zero or an integer from the range of from 1 to 4, R^{y} is selected from the group consisting of hydrogen atom, C1 to C3 alkyl group and (CH₂)_{c}SO₃⁻ group with c being an integer from the range of from 1 to 4,
   wherein at least one of R^{x}, R^{y}, R^{z} is a (CH₂)_{c}SO₃⁻ group,
   with the condition that, if R^{x} is a (CH₂)_{c}SO₃⁻ group with c being zero, then R^{z} is not a (CH₂)_{c}SO₃⁻ group with c being zero.

According to an embodiment of the azadibenzocyclooctyne derivative or salt thereof,
R¹, R² are each a —[(CH₂)ₐCR^{x}R^{y}]_{b}R^{z} group, wherein for each R¹, R² independently, a is either zero or an integer from the range of from 1 to 4, b is 1, R^{x}, R^{z} are selected from the group consisting of hydrogen atom, C1 to C3 alkyl group and (CH₂)_{c}SO₃⁻ group with c being either zero or an integer from the range of from 1 to 4, R^{y} is selected from the group consisting of hydrogen atom, C1 to C3 alkyl group and (CH₂)_{c}SO₃⁻ group with c being an integer from the range of from 1 to 4,
   wherein at least one of R^{x}, R^{y}, R^{z} is a (CH₂)_{c}SO₃⁻ group,
   with the condition that, if R^{x} is a (CH₂)_{c}SO₃⁻ group with c being zero, then R^{z} is not a (CH₂)_{c}SO₃⁻ group with c being zero.

According to an embodiment of the azadibenzocyclooctyne derivative or salt thereof,
R¹, R² are each a —[(CH₂)ₐCR^{x}R^{y}]_{b}R^{z} group, wherein for each R¹, R² independently, a is either zero or an integer from the range of from 1 to 4, b is 1, R^{x}, R^{z} are selected from the group consisting of hydrogen atom, C1 to C3 alkyl group and (CH₂)_{c}SO₃⁻ group with c being either zero or an integer from the range of from 1 to 4, R^{y} is a hydrogen atom, wherein at least one of R^{x}, R^{z} is a (CH₂)_{c}SO₃⁻ group;
   with the condition that, if R^{x} is a (CH₂)_{c}SO₃⁻ group with c being zero, then R^{z} is not a (CH₂)_{c}SO₃⁻ group with c being zero.

According to an embodiment of the azadibenzocyclooctyne derivative or salt thereof,
R¹, R² are each a —[(CH₂)ₐCR^{x}R^{y}]_{b}R^{z} group, wherein for each R¹, R² independently: a is either zero or an integer from the range of from 1 to 4, b is either zero or an integer from the range of from 1 to 3, R^{x}, R^{y}, R^{z} are selected from the group consisting of hydrogen atom and (CH₂)_{c}SO₃⁻ group, with c being either zero or an integer from the range of from 1 to 4,
   wherein at least one of R^{x}, R^{y}, R^{z} is a (CH₂)_{c}SO₃⁻ group with the condition:
   if R^{z} is a (CH₂)_{c}SO₃⁻ group with c being zero, then R^{x}, R^{y} are not both a (CH₂)_{c}SO₃⁻ group wherein c is zero, or
   if a is zero, then R^{x} and R^{y} are not both a (CH₂)_{c}SO₃⁻ group wherein c is zero.

According to an embodiment of the azadibenzocyclooctyne derivative or salt thereof, R¹, R² are the same and are both a —[(CH₂)ₐCR^{x}R^{y}]_{b}R^{z} group, wherein b is zero or 1. The index “a” and R^{x}, R^{y}, R^{z} are as defined above.

According to an embodiment of the azadibenzocyclooctyne derivative or salt thereof, R¹, R² are each a —[CR^{r}R^{s}]_{d}—R^{t} group, wherein for each R¹, R² independently: R^{r} is selected from the group consisting of hydrogen atom, hydroxyl group and —[CH(OH)]ₑ—H, R^{s} is either a hydrogen atom or a —[CH(OH)]_{f}—H group, and R^{t} is selected from the group consisting of hydrogen atom, C1 to C5 alkyl and —[CH(OH)]_{g}—H group, wherein each of d, e, f, g is independently an integer selected from the range of from 1 to 10, with the condition that if R^{t} is a hydrogen atom or a C1 to C5 alkyl group, then at least one of R^{r}, R^{s} is not a hydrogen atom.

According to an embodiment of the azadibenzocyclooctyne derivative or salt thereof, R¹, R² are the same and are each a —[CH(OH)]_{d}—H group, wherein d is an integer selected from the range of from 1 to 10, preferably from the range of from 1 to 5, more preferably d is 2 or 3.

According to an embodiment of the azadibenzocyclooctyne derivative or salt thereof, R⁵ is an activated carboxyl group and the activation group of R⁵ is selected from the group consisting of 4-nitrophenyl group, pentafluorophenyl group and N-succinimidyl group, preferably an N-succinimidyl group.

In cases where R⁵ is a carboxyl group, said group can be in situ activated by, for example, HATU (1-[bis(dimethylamino)methylene]-1H-1,2,3-triazolo[4,5-b]pyridinium 3-oxide hexafluorophosphate), HBTU (3-[bis(dimethylamino)methyliumyl]-3H-benzotriazol-1-oxide hexafluorophosphate), a carbodiimide, preferably selected from the group consisting of N,N′-diisopropyl carbodiimide (DIC), N,N′-dicyclohexyl carbodiimide (DCC) and 1-ethyl-3-(3-dimethyl amino propyl)carbodiimide (EDC), or a phosphonium salt, preferably benzotriazol-1-yloxytris(dimethylamino)phosphonium hexafluorophosphate (BOP) or benzotriazol-1-yloxytripyrrolidinophosphonium hexafluorophosphate (PyBOP).

According to an embodiment of the azadibenzocyclooctyne derivative or salt thereof, L is a structure —(CH₂)ₚ—(X)ₘ—(CH₂)_{q}—, wherein
p, q are independently an integer selected from the range of from 2 to 10;
X is selected from the group consisting of —C(═Y)—NH—, —NH—C(═Y)—, —C(═Y)—O— and —O—C(═Y)—, wherein Y is an oxygen atom or a sulphur atom;
m is zero or 1 [if m is zero, then X is absent and (CH₂)ₚ, (CH₂)_{q} are directly connected by a single bond].

According to an embodiment of the azadibenzocyclooctyne derivative or salt thereof, m is 1 and X is a —C(═O)—NH— group and/or wherein p, q are identical and are both an integer selected from the range of from 2 to 5, preferably 2 or 3.

According to an embodiment, the azadibenzocyclooctyne derivative or salt thereof has formula (Ia), (Ib) or (Ic):

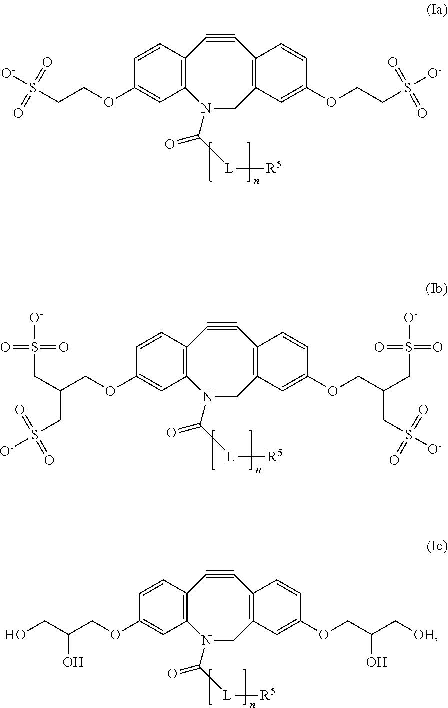

wherein L, n and R⁵ are as defined above.

According to an embodiment, the azadibenzocyclooctyne derivative or salt thereof has formula formula (Ia-1), (Ib-1) or (Ic-1):

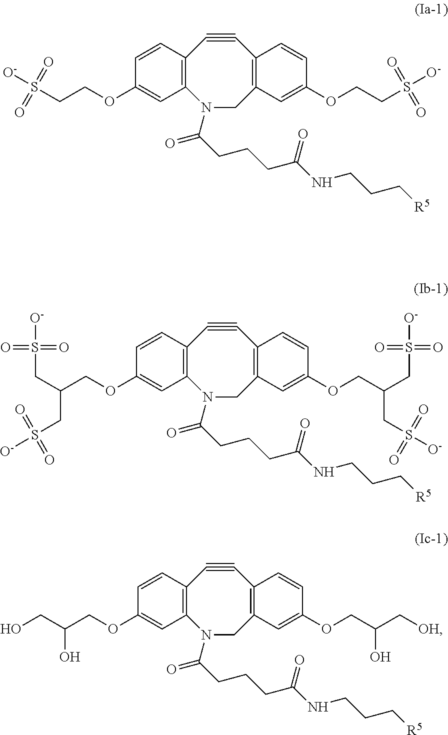

wherein R⁵ is as defined above.

According to an embodiment, the azadibenzocyclooctyne derivative or salt thereof has formula formula (Ia-1) or (Ib-1), preferably (Ia-1):

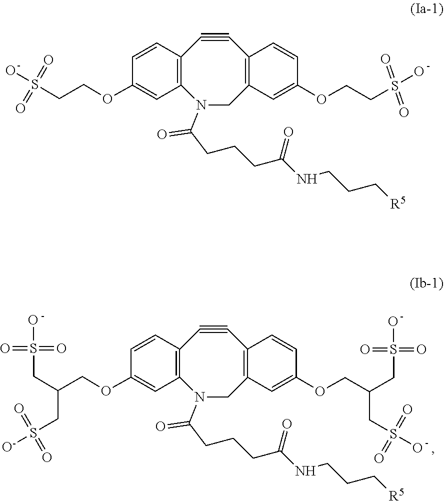

### 2^{nd} ASPECT—CONJUGATE

In a second aspect, the invention is related to a conjugate of formula (II)

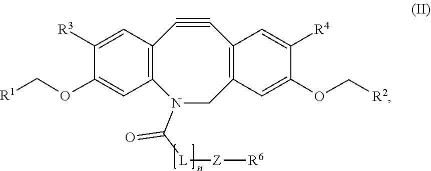

wherein L, R¹, R², R³, R⁴ and n are as defined above with respect to the first aspect for the azadibenzocyclooctyne derivative of formula (I) or salt thereof; and wherein
R⁶ is selected from the group consisting of fluorophore, fluorescence quencher, dye, hapten, tyramine, polyethylene glycol chain, polypropylene glycol chain, mixed polyethylene/polypropylene glycol chain, metal complex, radioactive isotope, active pharmaceutical ingredient, carbohydrate, solid phase, lipid, amino acid, oligopeptide, polypeptide, nucleotide, oligonucleotide and polynucleotide; and is preferably a metal complex; and
Z is selected from the group consisting of —C(═O)—O—, —C(═O)—NR⁷—, and —NR⁷—C(═Y)—, wherein R⁷ is a hydrogen atom or a C1-C5 alkyl group and Y is an oxygen atom or a sulphur atom, preferably an oxygen atom.

All definitions given above in the section related to the first aspect also apply with respect to the conjugate of the second aspect.

According to an embodiment of the conjugate of formula (II), R⁶ is selected from the group consisting of fluorophore, fluorescence quencher, dye, hapten, tyramine, metal complex, radioactive isotope, active pharmaceutical ingredient (drug), carbohydrate, solid phase, lipid, amino acid, oligopeptide, polypeptide, nucleotide, oligonucleotide and polynucleotide; and is preferably a metal complex, wherein a further linker is present or absent between Z and R⁶, which is preferably selected from the group consisting of alkyl chain, polyethylene glycol chain, polypropylene glycol chain and mixed polyethylene/polypropylene glycol chain.

A metal complex is preferably a Ruthenium(II)-or Iridium(III)-based complex, more preferably a Ruthenium(II)-or Iridium(III)-based electrochemiluminescent complex. Electrochemiluminescense (ECL) proved to be very useful in analytical applications as a highly sensitive and selective method. It combines analytical advantages of chemiluminescent analysis (absence of background optical signal) with ease of reaction control by applying electrode potential. In general Ruthenium(II) complexes, especially [Ru(bpy)₃]²⁺ (which releases a photon at ˜620 nm) regenerating with TPA (Tripropylamine) in liquid phase or liquid-solid interface are used as ECL-labels. Ruthenium(II) complexes, which are usable as ECL-labels, are described in WO 2003/002974 A2. Iridium(III) complexes, which are usable as ECL-labels, are described in WO 2012/107419 A1, WO 2012/107420 A1 and also in WO 2014/019709 A2 and WO 2014/019708 A1. Preferably, the Iridum(III) complex comprises Ir³⁺ and two substituted or unsubstituted 6-phenylphenanthridine ligands and optionally an, optionally modified, pyridine-2-carboxylic acid or 2-(1H-pyrazole-3-yl)pyridine. Especially in view of further bonding, the 2-(1H-pyrazole-3-yl)pyridine is modified with a reactive unit, for example, the 2-(1H-pyrazole-3-yl)pyridine ligand is substituted with a 3-alkyl carboxylic acid group, which may be activated by an NHS group for conjugation.

Further metal complexes suitable as labels for imaging and therapeutic purposes are well-known in the art, see for example, WO 2017/153574 A1.

Radioactive labels make use of radioisotopes (radionuclides), such as 3H, 11C, 14C, 18F, 32P, 35S, 64Cu, 68Gn, 86Y, 89Zr, 99TC, 111In, 123I, 124I, 125I, 131I, 133Xe, 177Lu, 211At, or 131Bi.

“Fluorophores” include rare earth chelates (europium chelates), fluorescein type labels including FITC, 5-carboxyfluorescein, 6-carboxyfluorescein; rhodamine type labels including TAMRA, lissamine, Texas Red; dansyl; cyanines; coumarines, phycocrythrins; and analogues thereof. The fluorescent labels can be conjugated to the azadibenzocyclooctyne derivatives of the invention using methods known to the person skilled in the art, preferably via formation of an amide bond. Fluorescent and non-fluorescent dyes and label reagents including also Alexa, Atto and DY dyes are commercially available, e.g., from Invitrogen/Molecular Probes (Eugene, Oregon, USA), ThermoFisher Scientific (Waltham, Massachusetts, USA), Sigma Aldrich, Atto-Tec GmbH (Siegen), Dyomics GmbH (Jena) and Pierce Biotechnology, Inc. (Rockford, Ill.). “Fluorescence quenchers”, such as for example, black hole quenchers (BHQs), are known to the skilled person. “Dyes” for example, azo dyes like dabcyl or dabsyl are also known to the skilled person.

A “hapten” is an organic molecule with a molecular weight of 100 to 2000 Dalton. In one embodiment the hapten has a molecular weight of 100 to 1000 Dalton. Usually an organic molecule of such molecular weight is not immunogenic or of comparatively low immunogenicity. A hapten can be rendered immunogenic by coupling it to a carrier molecule and anti-hapten antibodies can be generated according to standard procedures. In one embodiment the hapten may be selected from the group comprising sterols, bile acids, sexual hormones, corticoids, cardenolides, cardenolide-glycosides, bufadienolides, steroid-sapogenines and steroid alkaloids, cardenolides, cardenolide-glycosides and vitamines. Representatives of these substance classes are digoxigenin, digitoxigenin, gitoxigenin, strophanthidin, digoxin, digitoxin, ditoxin, strophanthin fluorescein, biotin, dinitrophenyl.

“Tyramine” is 4-(2-amino ethyl)phenol. When coupled to a label via the corresponding amide it is used as reagent for tyramide signal amplification by activation with horseradish peroxidase (HRP) , e.g. an antibody-HRP conjugate (see, for example, Perkin-Elmer, ThermoFisher).

An “oligopeptide” is a peptide which comprises in the range of from 2 to 9 amino acid residues. A “polypeptide” is a peptide which comprises at least 10 amino acid residues. In some embodiments, the peptide comprises at least 10 amino acid residues, or at least 20 amino acid residues. In some embodiments, the peptide comprises not more than 1000 amino acid residues, such as not more than 500 amino acid residues, for example not more than 100 amino acid residues. In some embodiments, the polypeptide is an enzyme or an antibody.

An “oligonucleotide” comprises in the range of from 2 to 9 covalently bonded nucleotide monomers. A “polynucleotide” comprises at least 10 covalently bonded nucleotide monomers. In some embodiments, the polynucleotide comprises not more than 1000 nucleotide monomers. The oligonucleotide and/or the polynucleotide is either single stranded or double stranded. The term oligonucleotide or polynucleotide is to be understood broadly and includes DNA and RNA as well as analogues and modifications thereof. An “analogue” may for example contain a substituted nucleotide carrying a substituent at the standard bases adenine, guanine, cytosine, thymine, uracil. Examples of such nucleosides comprising substituted nucleobases are: 5-substituted pyrimidines like 5-methyl-dC, aminoallyl-dU or -dC, 5-(aminocthyl-3-acrylimido)-dU, 5-propinyl-dU or -dC, 5-halogenated dU or dC; N-substituted pyrimidines like N4-ethyl-dC; N-substituted purines like N6-ethyl-dA, N2-ethyl-dG; 8-substituted purines like 8-[(6-amino-hex-1-yl)-amino]-dG or-dA, 8-halogenated dA or dG, 8-alkyl-dG or-dA; and 2-substituted dA like 2-amino-dA. An “analogue” may contain a nucleotide or a nucleoside analogue. I.e. the naturally occurring nucleobases can be exchanged by using nucleobase analogues like 5-nitroindol-d-riboside; 3-nitropyrrole-d-riboside, deoxyinosine (dI), deoxyxanthosine (dX); 7-deaza-dG, -dA, -dI or -dX; 7-deaza-8-aza-dG, -dA, -dI or -dX; 8-aza-dA, -dG, -dI or -dX; d-formycin; pseudo-dU; pseudo-iso-dC; 4-thio-dT; 6-thio-dG; 2-thio-dT; iso-dG; 5-methyl-iso-dC; N8-linked 8-aza-7-deaza-dA; 5,6-dihydro-5-aza-dC; and etheno-dA or pyrrolo-dC. As obvious to the skilled artisan, in case of double strands, the nucleobase in the complementary strand has to be selected in such manner that duplex formation is specific. If, for example, 5-methyl-iso-dC is used in one strand (e.g. (a)) iso-dG has to be in the complementary strand (e.g. (a′)). In an “analogue”, the oligo-/polynucleotide backbone may be modified to contain substituted sugar residues, sugar analogues, modifications in the internucleoside phosphate moiety, and/or be a PNA. An oligonucleotide may for example contain a nucleotide with a substituted deoxyribose like 2′-methoxy, 2′-fluoro, 2′-methylseleno, 2′-allyloxy, 4′-methyl-dN (wherein N is a nucleobase, e.g., A, G, C, T or U).

In some preferred embodiments, R⁶ is an oligonucleotide or a polynucleotide, preferably a single stranded DNA (ssDNA) having preferably in the range of from 4 to 12 nucleotides, wherein more preferably all nucleotides are non-natural nucleotides, i.e. comprise nucleotide analogues or nucleoside analogues.

In some preferred embodiments, R⁶ is an oligonucleotide or a polynucleotide, preferably an LNA gapmer (“LNA” means locked nucleic acid, a “gapmer” is a short DNA antisense oligonucleotide structure with RNA-like segments on both sides of the sequence; see P. H. Hagedorn et al., Drug Discovery Today 2018, 23(1), 101-114).

In some preferred embodiments, R⁶ is an oligonucleotide or a polynucleotide, preferably an beta-L-LNA single strand (“beta-L-LNA” means the L-configured stereoisomer of LNA, see WO 2019/243391 A1, WO 2020/245377 A1).

Sugar analogues are for example xylose; 2′,4′-bridged ribose like (2′-O, 4′-C-methylene)-bridged ribose (oligomer known as LNA) or (2′-O, 4′-C-ethylene)-bridged ribose (oligomer known as ENA); L-ribose, L-d-ribose, hexitol (oligomer known as HNA); cyclohexenyl (oligomer known as CeNA); altritol (oligomer known as ANA); a tricyclic ribose analogue where C3′ and C5′ atoms are connected by an ethylene bridge that is fused to a cyclopropane ring (oligomer known as tricycloDNA); glycerol (oligomer known as GNA); glucopyranose (oligomer known as homo-DNA); carbaribose (with a cyclopentan instead of a tetrahydrofuran subunit); hydroxymethyl-morpholine (oligomers known as morpholino DNA).

A great number of modifications comprising a modified internucleosidic phosphate moiety are also known not to interfere with hybridization properties and such backbone modifications can also be combined with substituted nucleotides or nucleotide analogues. Examples are phosphorothioate, phosphorodithioate, phosphoramidate and methylphosphonate oligonucleotides.

PNA (having a backbone without phosphate and d-ribose) can also be used as a DNA analogue.

A “solid phase” is typically glass or a polymer, the most commonly used polymers being cellulose, polyacrylamide, nylon, polystyrene, polyvinyl chloride, or polypropylene. As the skilled artisan will appreciate a solid phase can either by its nature contain an aldehyde functionality or can be chemically modified to introduce an aldehyde group. As further evident, a solid phase can be coated with any of a polypeptide, a carbohydrate, a nucleotide and a nucleic acid. In some embodiments, the solid phase is coated with streptavidin. The solid phase may be in the form of tubes, beads, or discs of microplates. In one embodiment the solid phase is a paramagnetic bead based on glass or any of the above mentioned polymers.

A “carbohydrate” is a biological molecule consisting of carbon (C), hydrogen (H) and oxygen (O) atoms, usually with a hydrogen-oxygen atom ratio of 2:1 (as in water); in other words, with the empirical formula Cᵥ(H₂O)_{w} (where v usually is the same as w). Some exceptions exist (v is different from w); for example, deoxyribose, a sugar component of DNA, has the empirical formula C5H10O4. Carbohydrates are technically hydrates of carbon; structurally it is more accurate to view them as polyhydroxy aldehydes and ketones.

The term carbohydrate is most common in biochemistry, where it is a synonym of ‘saccharide’, a group of molecules that includes sugars, starch, and cellulose. In one embodiment the carbohydrate is selected from sugars, starch, and cellulose.

The term “antibody” herein is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity.

An “isolated” antibody is one which has been identified and separated and/or recovered from a component of its natural environment. Contaminant components of its natural environment are materials which would interfere with research, diagnostic or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. Isolated antibody includes the antibody in situ within recombinant cells since at least one component of the antibody's natural environment will not be present. Ordinarily, however, isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues are believed to form an interface between the light-chain and heavy-chain variable domains.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light-chain and the heavy-chain variable domains.

The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, MD (1991)). The constant domains are not involved directly in the binding of an antibody to an antigen, but exhibit various effector functions, such as participation of the antibody in antibody-dependent cellular toxicity.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequences of the constant domains of their heavy chains, antibodies (immunoglobulins) can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The subunit structures and three-dimensional configurations of different classes of immunoglobulins are well known and described generally in, for example, Abbas et al., Cellular and Mol. Immunology, 4th ed., W.B. Saunders, Co. (2000). An antibody may be part of a larger fusion molecule, formed by covalent or non-covalent association of the antibody with one or more other proteins or peptides.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody, preferably comprising the antigen-binding region thereof. Examples of antibody fragments include Fab, Fab′, F(ab′)2, and Fv fragments; diabodies; linear antibodies; single-chain antibody molecules; and multispecific antibodies formed from antibody fragments.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields a F(ab′)2 fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy- and one light-chain variable domain can be covalently linked by a flexible peptide linker such that the light and heavy chains can associate in a “dimeric” structure analogous to that in a two-chain Fv species. It is in this configuration that the three HVRs of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy-and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody-hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

“Single-chain Fv” or “scFv” antibody fragments comprise the VH and VL domains of an antibody, wherein these domains are present in a single polypeptide chain. Generally, the scFv polypeptide further comprises a polypeptide linker between the VH and VL domains that enables the scFv to form the desired structure for antigen binding. For a review of scFv, sec, e.g., Plueckthun, In: The Pharmacology of Monoclonal Antibodies, Vol. 113, Rosenburg and Moore (eds.), Springer-Verlag, New York (1994) pp. 269-315.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 0404 097 A1; WO 1993/01161 A1; Hudson, P. J. et al., Nat. Med. 9 (2003) 129-134; and Holliger, P. et al., PNAS USA 90 (1993) 6444-6448. Triabodies and tetrabodies are also described in Hudson, P. J. et al., Nat. Med. 9 (2003) 129-134.

The term “monoclonal antibody” as used herein refers to an antibody obtained from a population of substantially homogeneous antibodies, i.e., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations, which typically include different antibodies directed against different determinants (epitopes), each monoclonal antibody of a monoclonal-antibody preparation is directed against a single determinant on an antigen. In addition to their specificity, monoclonal-antibody preparations are advantageous in that they are typically uncontaminated by other immunoglobulins.

A “lipid” is a, preferably natural, substance that is completely or at least largely insoluble in water (hydrophobic), but which dissolve very well in hydrophobic (or lipophilic) solvents such as hexane due to their low polarity. The lipid is preferably selected from the group consisting of fatty acid, triglyceride (fat and fatty oil), wax, phospholipid, sphingolipid, lipopolysaccharide and isoprenoid. In some embodiments, the isoprenoid is a steroid. A “steroid” is a derivative of the hydrocarbon sterane.

A “active pharmaceutical ingredient” includes any pharmaceutically active chemical or biological compound and any pharmaceutically acceptable salt thereof and any mixture thereof, that provides some pharmacologic effect and is used for treating or preventing a pathological condition or a disease. Preferably, especially in the context of antibody-drug-conjugates (ADCs), the active pharmaceutical ingredient is a toxin or cytotoxin such as amanitin or maitansin.

The meaning of the terms polyethylene glycol chain, polypropylene glycol chain, mixed polyethylene/polypropylene glycol chain are clear to the skilled person. Preferably, each of these chain types has an average molecular weight in the range of from 100 to 10,000 Da.

According to an embodiment, the conjugate has formula (IIa), (IIb) or (IIc):

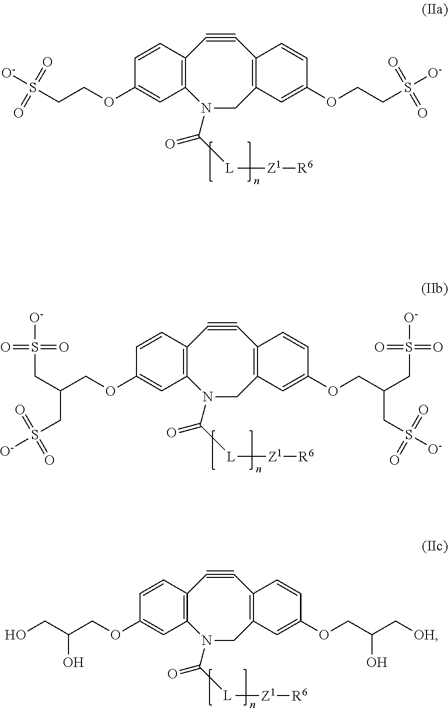

wherein L, n and R⁶ are as defined above and Z¹ is a —C(═O)—O-group or a —C(═O)—NH-group.

According to an embodiment, the conjugate has formula (IIa-1), (IIa-2), (IIb-1), (IIb-2), (IIc-1) or (IIc-2):

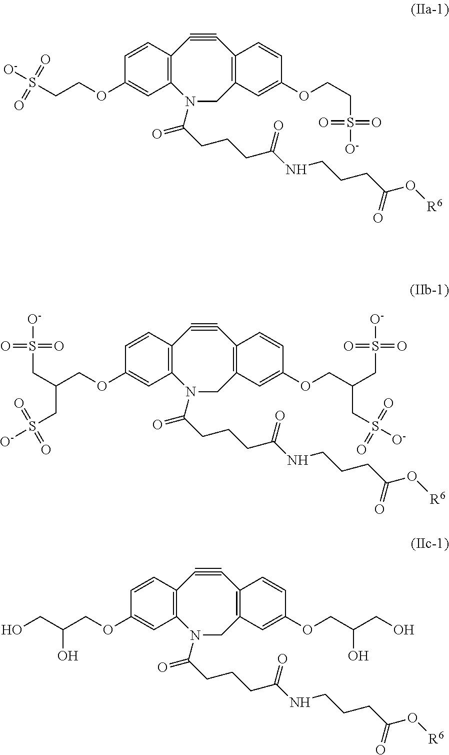

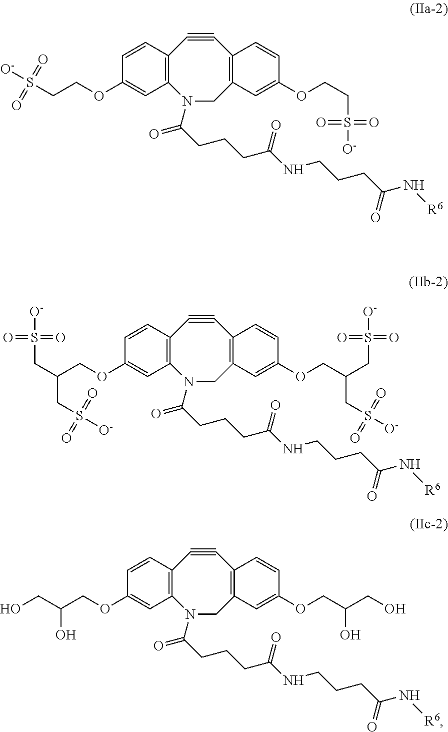

wherein R⁶ in (IIa-1), (IIa-2), (IIb-1), (IIb-2), (IIc-1) or (IIc-2) is as defined above.

### 3^{rd} ASPECT—METHOD FOR MODIFICATION OF A TARGET MOLECULE

In a third aspect, the invention is related to a method for the modification of a target molecule, wherein a conjugate according to the second aspect is reacted with a target molecule comprising a 1,3-dipole group or a 1,3-(hetero)diene group.

The reaction is done via a strain-promoted cycloaddition of the cycloalkyne derivatives of the invention, for example, with an azide (SPAAC). The reaction of a cyclooctyne with a 1,3-(hetero)diene is also known as a (hetero) Diels-Alder reaction. These reactions are also referred to as metal-free click reactions. The alkoxy substituted core increases the speed of the cycloaddition compared to commercially available derivatives. Using a conjugate according to the second aspect for the reaction with the target molecule circumvents the need of copper catalysis for the cycloaddition, i.e. the reaction is preferably done in the absence of a copper catalysis, more preferably in the absence of any catalyst.

Regarding the conjugate, all definitions apply as described above in the section related to the second aspect.

According to an embodiment of the method for the modification of a target molecule, the 1,3-dipole group is selected from the group consisting of azide, nitrone, diazoalkane, diazoacetamide and nitrile oxide and is preferably an azide.

According to an embodiment of the method for the modification of a target molecule, the 1,3-diene group is selected from the group consisting of 1,3-butadiene, 1,3-cyclopentadiene, 1,3-cyclohexadiene, furan, and a pyrrole.

According to an embodiment of the method for the modification of a target molecule, the 1,3-heterodiene group is selected from the group consisting of tetrazine, 1-oxa-1,3-butadiene, 1-aza-1,3-butadiene, 2-aza-1,3-butadiene, and 3-aza-1,3-butadiene.

According to an embodiment of the method for the modification, the target molecule is selected from the group consisting of fluorophore, fluorescence quencher, dye, hapten, tyramine, polyethylene glycol chain, polypropylene glycol chain, mixed polyethylene/polypropylene glycol chain, metal complex, radioactive isotope, active pharmaceutical ingredient, carbohydrate, solid phase, lipid, amino acid, oligopeptide, polypeptide, nucleotide, oligonucleotide, and polynucleotide; and is preferably a polypeptide, more preferably an antibody, more preferably a modified antibody having a 1,3-dipole group, more preferably a modified antibody having an azide group.

The meaning of the terms fluorophore, fluorescence quencher, dye, hapten, tyramine, polyethylene glycol chain, polypropylene glycol chain, mixed polyethylene/polypropylene glycol chain, metal complex, radioactive isotope, steroid, active pharmaceutical ingredient, carbohydrate, solid phase, amino acid, oligopeptide, polypeptide, nucleotide, oligonucleotide, and polynucleotide are as explained above.

In some embodiments, R⁶ of the conjugate of formula (II), is selected from the group consisting of fluorophore, fluorescence quencher, dye, hapten, tyramine, metal complex, active pharmaceutically compound (drug), solid phase, oligonucleotide, polynucleotide, lipid, polypeptide, especially enzyme or antibody, oligopeptide, polypeptide and polyethylene glycol, wherein preferably the target molecule is selected from the group consisting of antibody, oligonucleotide and polynucleotide. Regarding the oligo-and polynucleotides, it is preferred in some embodiments that these are antisense oligonucleotides like LNA gapmers or L-LNA single strands. Thus, the conjugates obtained or obtainable from a reaction of the conjugate according to the second aspect and a target molecule-herein also abbreviated as “target molecule conjugates” (see 5^{th} aspect below)-as described above may comprise but are not limited to antibody metal complex conjugates, antibody drug conjugates, antibody oligonucleotide conjugates, antibody solid phase conjugates, antibody fluorophore conjugates, antibody fluorescence quencher conjugates, antibody hapten conjugates, antibody enzyme conjugates, antibody antibody conjugates, antibody polyethylene glycol conjugates, oligonucleotide oligo- or polypeptide conjugates, oligonucleotide lipid conjugates, oligonucleotide solid phase conjugates, tyramide dye conjugate and the like. Examples of antibody oligonucleotide conjugates are but not limited to antibodies modified with antisense oligonucleotides like LNA gapmers (P. H. Hagedorn et al., Drug Discovery Today 2018, 23(1), 101-114) or L-LNA single strands (WO 2019/243391 A1, WO 2020/245377 A1). Antibody oligonucleotide conjugates and their use for targeted delivery are for example described in WO 2020/247738 A1. The antibody is as defined above and is more preferably selected from IgG and Fab fragment.

### 4^{th} ASPECT—USE OF THE CONJUGATE

A fourth aspect of the invention is directed to the use of a conjugate according to the second aspect for bioorthogonal labeling and/or modification of a target molecule.

All definitions given above in the section related to the second aspect also apply with respect to the use described here. Regarding options for or bioorthogonal labeling and/or modification of a target molecule, all defintions apply as given above in the section related to the third aspect.

### 5^{th} ASPECT—MODIFIED TARGET MOLECULE

In a fifth aspect, the invention relates to a modified target molecule comprising the reaction product of a conjugate according to the second aspect and a target molecule comprising a 1,3-dipole group or a 1,3-(hetero)diene group, obtained or obtainable from the method of the third aspect.

All definitions given above in the section related to the second aspect also apply with respect to the use described here. Regarding options for or bioorthogonal labeling and/or modification of a target molecule, all definitions apply as given above in the section related to the third aspect.

The reaction products of a conjugate according to the second aspect and a target molecule comprising a 1,3-dipole group or a 1,3-(hetero)diene group, which are also abbreviated as “target molecule conjugates” enable a reduction of the background signal conjugates in comparison to that of reference DBCO conjugates according to the state of the art. Without being bound to that theory, it is assumed that said reduction of the background signal can be attributed to hydrophilization (due to sulfonation or hydroxylation) of the otherwise hydrophobic moieties.

Stability tests revealed a higher recovery of the ECL-signal for inventive target molecule conjugates compared to the reference DBCO conjugates according to the state of the art.

### 6^{th} ASPECT—KIT

A sixth aspect of the invention is directed to a kit comprising a modified target molecule according to the fifth aspect as detector reagent, wherein the target molecule is preferably an antibody and R⁶ is preferably a metal complex, and a suitable capture reagent.

All definitions given above in the section related to the second aspect also apply with respect to the use described here. Regarding options for or bioorthogonal labeling and/or modification of a target molecule, all defintions apply as given above in the section related to the third aspect.

As indicated above, the target molecule is preferably an antibody, wherein the antibody is preferably as defined above in the second related to the third aspect and is more preferably selected from IgG and Fab fragment. As indicated above, the metal complex is preferably a Ruthenium(II) based complex or an Iridium(III) based complex as defined above in the section related to the third aspect. A solid phase is preferably also part of the kit, wherein said solid phase is as defined above in the section related to the third aspect and has preferably a coating with streptavidin.

The present invention is further illustrated by the following embodiments and combinations of embodiments as indicated by the respective dependencies and back-references. In particular, it is noted that in each instance where a range of embodiments is mentioned, for example in the context of a term such as “The process of any one of embodiments 1 to 4”, every embodiment in this range is meant to be explicitly disclosed for the skilled person, i.e. the wording of this term is to be understood by the skilled person as being synonymous to “The process of any one of embodiments 1, 2, 3, and 4”.

1. An azadibenzocyclooctyne derivative according to formula (I) or a salt thereof,

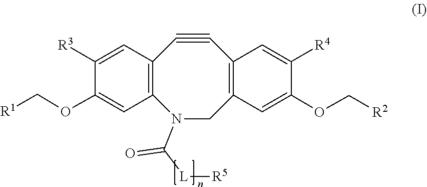

wherein
R¹, R² are independently selected from the group consisting of
   —[(CH₂)ₐCR^{x}R^{y}]_{b}R^{z} group, wherein a is either zero or an integer from the range of from 1 to 4, b is either zero or an integer from the range of from 1 to 3, R^{x}, R^{y}, R^{z} are independently selected from the group consisting of hydrogen atom, C1 to C3 alkyl group and (CH₂)_{c}SO₃⁻ group, with c being either zero or an integer from the range of from 1 to 4, wherein at least one of R^{x}, R^{y}, R^{z} is a (CH₂)_{c}SO₃⁻ group with the condition:
   if R^{z} is a (CH₂)_{c}SO₃⁻ group with c being zero, then R^{x}, R^{y} are not both a (CH₂)_{c}SO₃⁻ group wherein c is zero, or
   if a is zero, then R^{x} and R^{y} are not both a (CH₂)_{c}SO₃⁻ group wherein c is zero;
   and
   —[CR^{r}R^{s}]_{d}—R^{t} group, wherein d is an integer selected from the range of from 1 to 10, R^{r} is selected from the group consisting of hydrogen atom, hydroxyl group and —[CR′(OH)]ₑ—H group, R^{s} is either a hydrogen atom or a —[CR″(OH)]_{f}—H group, and R^{t} is selected from the group consisting of hydrogen atom, C1 to C5 alkyl group and —[CR″′(OH)]_{g}—H group, wherein each R′, R″, R″′ is independently either a hydrogen atom or a —[CH(OH)]ₕ—H group, and each of d, e, f, g and h is independently an integer selected from the range of from 1 to 10, with the condition that if R^{t} is a hydrogen atom or a C1 to C5 alkyl group, then at least one of R^{r}, R^{s} is not a hydrogen atom;
R³, R⁴ are independently selected from the group consisting of hydrogen atom, C1-C3-alkyl group, halogen atom and —O—C1-C3-alkyl group; and
R⁵ is selected from the group consisting of carboxyl group, activated carboxyl group and —NHR^{5a} group, wherein R^{5a} is a hydrogen atom or a C1-C5 alkyl group;
L comprises a chain of covalently bonded atoms forming a backbone and having a length in the range of from 1 to 100 atoms (linker); and
n is either zero or 1 if R⁵ is a carboxyl group or an activated carboxyl group or n is 1 if R⁵ is a —NHR^{5a} group.
   2. The azadibenzocyclooctyne derivative or salt thereof of any one of embodiment 1, wherein R³, R⁴ are independently a hydrogen atom or a methyl group, preferably R³, R⁴ are identical and are each a hydrogen atom.
   3. The azadibenzocyclooctyne derivative or salt thereof of embodiment 1 or 2, wherein
R¹, R² are each a —[(CH₂)ₐCR^{x}R^{y}]_{b}R^{z} group, wherein for each R¹, R² independently, a is either zero or an integer from the range of from 1 to 4, b is either zero or an integer from the range of from 1 to 3, R^{x}, R^{y}, R^{z} are selected from the group consisting of hydrogen atom, C1 to C3 alkyl group and (CH₂)_{c}SO₃⁻ group, with c being either zero or an integer from the range of from 1 to 4, wherein at least one of R^{x}, R^{y}, R^{z} is a (CH₂)_{c}SO₃⁻ group with the condition:
   if R^{z} is a (CH₂)_{c}SO₃⁻ group with c being zero, then R^{x}, R^{y} are not both a (CH₂)_{c}SO₃⁻ group wherein c is zero, or
   if a is zero, then R^{x} and R^{y} are not both a (CH₂)_{c}SO₃⁻ group wherein c is zero.
      4. The azadibenzocyclooctyne derivative or salt thereof of any one of embodiments 1 to 3, wherein
R¹, R² are each a —[(CH₂)ₐCR^{x}R^{y}]_{b}R^{z} group, wherein for each R¹, R² independently: a is either zero or an integer from the range of from 1 to 4, b is either zero or an integer from the range of from 1 to 3, R^{x}, R^{y}, R^{z} are selected from the group consisting of hydrogen atom and (CH₂)_{c}SO₃⁻ group, with c being either zero or an integer from the range of from 1 to 4,
   wherein at least one of R^{x}, R^{y}, R^{z} is a (CH₂)_{c}SO₃⁻ group with the condition:
   if R^{z} is a (CH₂)_{c}SO₃⁻ group with c being zero, then R^{x}, R^{y} are not both a (CH₂)_{c}SO₃⁻ group wherein c is zero, or
   if a is zero, then R^{x} and R^{y} are not both a (CH₂)_{c}SO₃⁻ group wherein c is zero.
      5. The azadibenzocyclooctyne derivative or salt thereof of any one of embodiments 1 to 4, wherein R¹, R² are the same and are both a —[(CH₂)ₐCR^{x}R^{y}]_{b}R^{z} group, wherein b is zero or 1.
      6. The azadibenzocyclooctyne derivative or salt thereof of any one of embodiments 1 to 2, wherein R¹, R² are each a —[CR^{r}R^{s}]_{d}—R^{t} group, wherein for each R¹, R² independently: R^{r} is selected from the group consisting of hydrogen atom, hydroxyl group and —[CH(OH)]ₑ—H, R^{s} is either a hydrogen atom or a —[CH(OH)]_{f}—H group, and R^{t} is selected from the group consisting of hydrogen atom, C1 to C5 alkyl and —[CH(OH)]_{g}H group, wherein each of d, e, f, g is independently an integer selected from the range of from 1 to 10, with the condition that if R^{t} is a hydrogen atom or a C1 to C5 alkyl group, then at least one of R^{t}, R^{s} is not a hydrogen atom.
      7. The azadibenzocyclooctyne derivative or salt thereof of any one of embodiments 1, 2 or 6, wherein R¹, R² are the same and are each a —[CH(OH)]_{d}—H group, wherein d is an integer selected from the range of from 1 to 10, preferably from the range of from 1 to 5, more preferably d is 2 or 3.
      8. The azadibenzocyclooctyne derivative or salt thereof of any one of embodiments 1 to 7, wherein R⁵ is an activated carboxyl group and the activation group of R⁵ is selected from the group consisting of 4-nitrophenyl group, pentafluorophenyl group and N-succinimidyl group, preferably an N-succinimidyl group.
      9. The azadibenzocyclooctyne derivative or salt thereof of any one of embodiments 1 to 8, wherein L is a structure —(CH₂)ₚ—(X)ₘ—(CH₂)_{q}—, wherein
p, q are independently an integer selected from the range of from 2 to 10;
X is selected from the group consisting of —C(═Y)—NH—, —NH—C(═Y)—, —C(═Y)—O— and —O—C(═Y)—, wherein Y is an oxygen atom or a sulphur atom;
m is zero or 1 [if m is zero, then X is absent and (CH₂)ₚ, (CH₂)_{q} are directly connected by a single bond].
   10. The azadibenzocyclooctyne derivative or salt thereof of embodiment 9, wherein m is 1 and X is a —C(═O)—NH-group and/or wherein p, q are identical and are both an integer selected from the range of from 2 to 5, preferably 2 or 3.
   11. The azadibenzocyclooctyne derivative or salt thereof of any one of embodiments 1 to 10 having formula (Ia), (Ib) or (Ic):

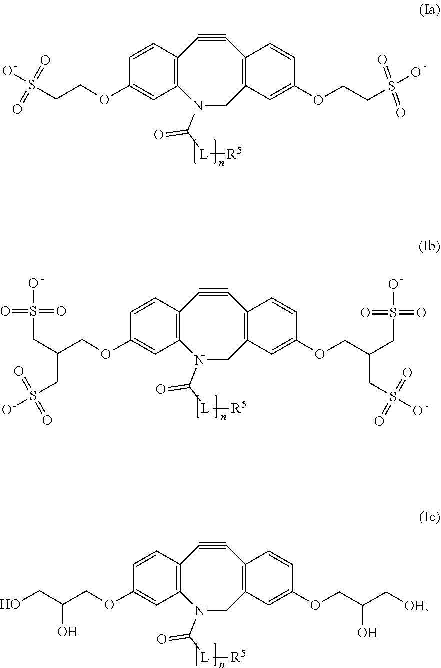

wherein L, n and R⁵ are as defined in any one of embodiments 1 to 9.
   12. The azadibenzocyclooctyne derivative or salt thereof of any one of embodiments 1 to 11 having formula (Ia-1), (Ib-1) or (Ic-1):

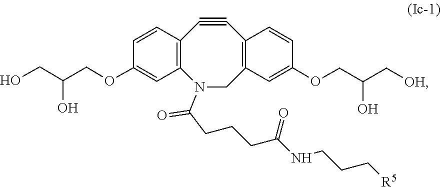

wherein R⁵ is as defined in any one of embodiments 1 to 9.
   13. The azadibenzocyclooctyne derivative or salt thereof of any one of embodiments 1 to 12 having formula (Ia-1) or (Ib-1), preferably (Ia-1):

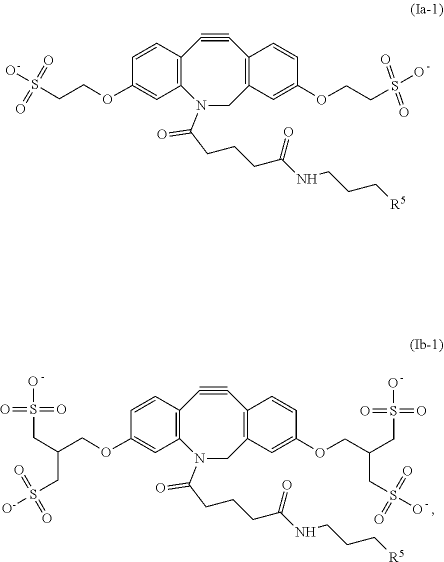

14. A conjugate of formula (II)

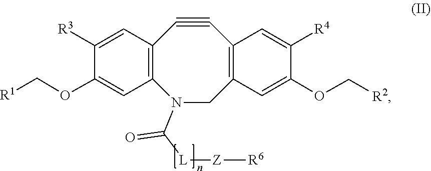

wherein L, R¹, R², R³, R⁴ and n are as defined in any one of embodiments 1 to 13 for the azadibenzocyclooctyne derivative of formula (I) or salt thereof; and wherein
R⁶ is selected from the group consisting of fluorophore, fluorescence quencher, dye, hapten, tyramine, polyethylene glycol chain, polypropylene glycol chain, mixed polyethylene/polypropylene glycol chain, metal complex, radioactive isotope, active pharmaceutical ingredient, carbohydrate, solid phase, lipid, amino acid, oligopeptide, polypeptide, nucleotide, oligonucleotide and polynucleotide; and is preferably a metal complex; and
Z is selected from the group consisting of —C(═O)—O—, —C(═O)—NR⁷—, and —NR⁷—C(═Y)—, wherein R⁷ is a hydrogen atom or a C1-C5 alkyl group and Y is an oxygen atom or a sulphur atom, preferably an oxygen atom.
   15. A conjugate of formula (II) according to embodiment 14, wherein R⁶ is selected from the group consisting of fluorophore, fluorescence quencher, dye, hapten, tyramine, metal complex, radioactive isotope, active pharmaceutical ingredient (drug), carbohydrate, solid phase, lipid, amino acid, oligopeptide, polypeptide, nucleotide, oligonucleotide and polynucleotide; and is preferably a metal complex, wherein a further linker is present or absent between Z and R⁶, which is preferably selected from the group consisting of alkyl chain, polyethylene glycol chain, polypropylene glycol chain and mixed polyethylene/polypropylene glycol chain.
   16. The conjugate of embodiment 14 or 15, having formula (IIa), (IIb) or (IIc):

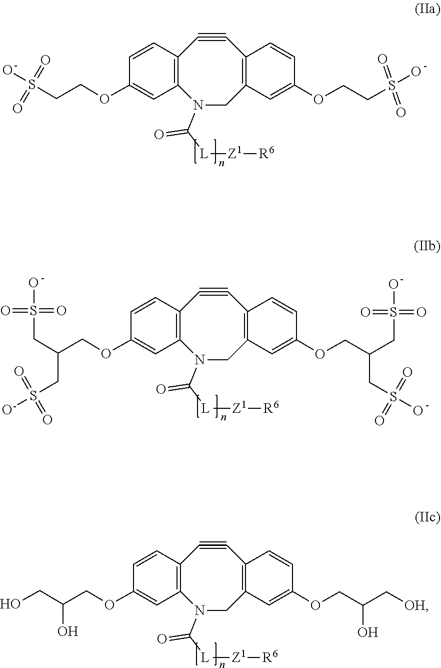

wherein L, n and R⁶ are as denned in embodiment 14 or 15 and Z¹ is a —C(═O)—O-group or a —C(═O)—NH-group.
   17. The conjugate of any one of embodiments 14 to 16, having formula (IIa-1), (IIa-2), (IIb-1), (IIb-2), (IIc-1) or (IIc-2):

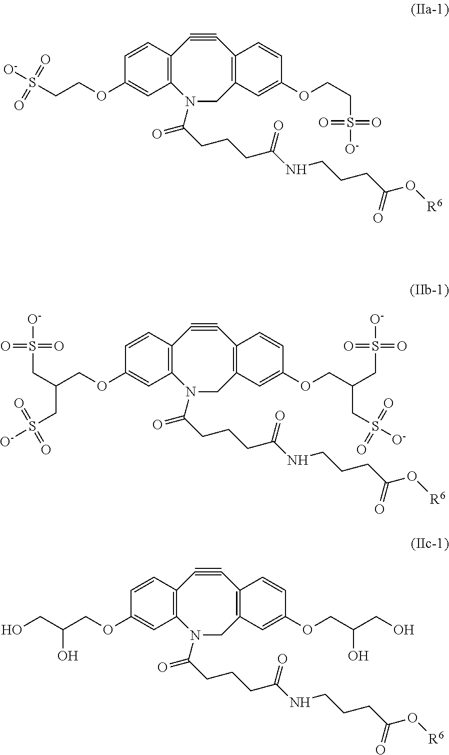

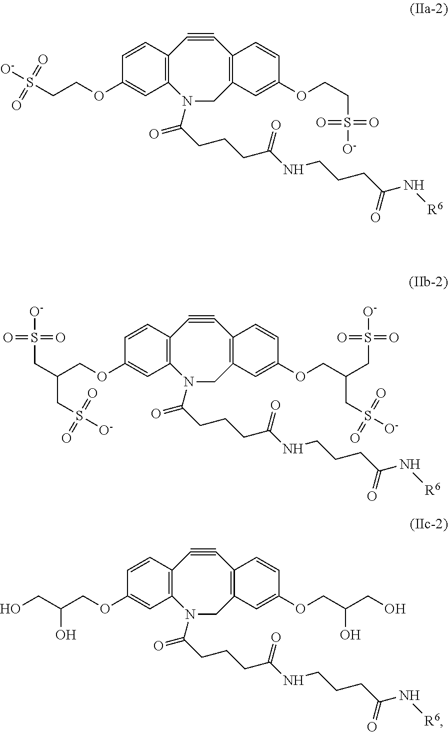

wherein R⁶ in (IIa-1), (IIa-2), (IIb-1), (IIb-2), (IIc-1) or (IIc-2) is as defined in any one of embodiments 14 to 16.
   18. A method for the modification of a target molecule, wherein a conjugate according to any one of embodiments 14 to 17 is reacted with a target molecule comprising a 1,3-dipole group or a 1,3-(hetero)diene group.
   19. The method according to embodiment 18, wherein the 1,3-dipole group is selected from the group consisting of azide, nitrone, diazoalkane, diazoacetamide and nitrile oxide and is preferably an azide.
   20. The method according to embodiment 18, wherein the 1,3-diene group is selected from the group consisting of 1,3-butadiene, 1,3-cyclopentadiene, 1,3-cyclohexadiene, furan, and a pyrrole.
   21. The method according to embodiment 18, wherein the 1,3-heterodiene group is selected from the group consisting of tetrazine, 1-oxa-1,3-butadiene, 1-aza-1,3-butadiene, 2-aza-1,3-butadiene, and 3-aza-1,3-butadiene.
   22. The method according to any one of embodiments 18 to 21, wherein the target molecule is selected from the group consisting of fluorophore, fluorescence quencher, dye, hapten, tyramine, polyethylene glycol chain, polypropylene glycol chain, mixed polyethylene/polypropylene glycol chain, metal complex, radioactive isotope, active pharmaceutical ingredient, carbohydrate, solid phase, lipid, amino acid, oligopeptide, polypeptide, nucleotide, oligonucleotide, and polynucleotide; and is preferably a polypeptide, more preferably an antibody, more preferably a modified antibody having a 1,3-dipole group, more preferably a modified antibody having an azide group.
   23. Use of a conjugate according to any one of embodiments 14 to 17 for bioorthogonal labeling and/or modification of a target molecule.
   24. A modified target molecule comprising the reaction product of a conjugate according to any one of embodiments 14 to 17 and a target molecule comprising a 1,3-dipole group or a 1,3-(hetero)diene group, obtained or obtainable from the method of any one of embodiments 18 to 22.
   25. A kit comprising a modified target molecule according to embodiment 24 as detector reagent, wherein the target molecule is preferably an antibody and R⁶ is preferably a metal complex, and a suitable capture reagent.

### EXAMPLES

The following Examples shall merely illustrate the invention. Whatsoever, they shall not be construed as limiting the scope of the invention.

### Experimental Procedures

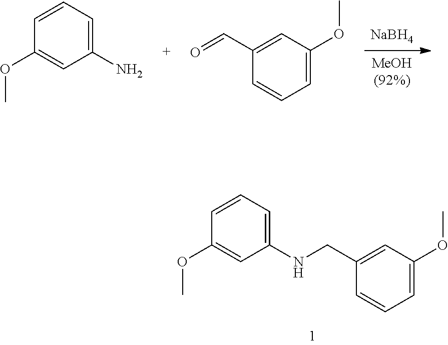

Aniline 1: A solution of m-anisidine (2.48 ml, 22.0 mmol) and m-anisaldehyde (2.44 ml, 20 mmol) in MeOH (200 ml) was stirred at room temperature for 1.5 h before NaBH₄ (2.26 g, 60.0 mmol) was added and the reaction was stirred at that temperature for another 1.5 h. Water (100 ml) was added and the mixture was extracted with EtOAc. The combined organic phases were washed with 1M NaOH, water and brine, dried over MgSO₄ and concentrated under reduced pressure. Purification of the resultant residue by flash column chromatography (hexane:EtOAc=5:1, R_{f}=0.3) gave aniline 1 (4.47 g, 18.4 mmol, 92%) as a yellowish oil.

R_{f}=0.3 [hexane/EtOAc, 5:1].

¹H NMR (400 MHZ, CDCl₃) δ=7.25 (t, J=7.84 Hz, 1H), 7.07 (t, J=8.09 Hz, 1H), 6.95 (dd, J=7.53, 0.63 Hz, 1H), 6.92 (m, 1H), 6.81 (m, 1H), 6.27 (dddd, J=11.26, 8.13, 2.32, 0.75 Hz, 2H), 6.19 (t, J=2.26 Hz, 1H), 4.29 (s, 2H), 4.05 (brs, 1H), 3.79 (s, 3H), 3.75 (s, 3H) ppm.

¹³C NMR (150 MHz, CDCl₃): δ=180.8, 159.9, 149.5, 141.0, 130.0, 129.6, 119.7, 113.0, 112.7, 106.0, 102.7, 98.9, 55.2, 55.1, 48.3 ppm.

MS (ESI): calcd. for C₁₅H₁₈NO₂⁺: 244.1 [M+H]⁺
found: 244.4 [M+H]⁺.

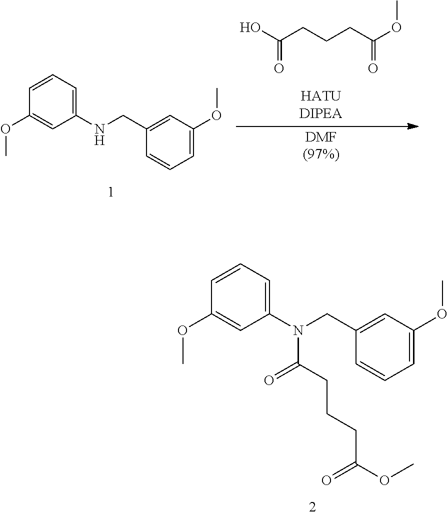

Acylamine 2: DIPEA (4.31 ml, 24.7 mmol) was added to a solution of aniline 1 (3.00 g, 12.3 mmol), mono methyl glutarate (2.01 ml, 16.7 mmol) and HATU (6.35 g, 16.7 mmol) in DMF (31 ml) and the reaction was allowed to stir at room temperature for 3 d. The mixture was diluted with EtOAc, washed with 1M HCl, water and brine, dried over MgSO₄ and concentrated under reduced pressure. Purification of the resultant residue by flash column chromatography (EtOAc:hexane=1:1) gave acylamine 2 (4.42 g, 11.9 mmol, 97%) as a pale yellow oil.

R_{f}=0.4 [hexane/EtOAc, 1:1].

¹H NMR (400 MHZ, CDCl₃) δ=7.19 (m, 2H), 6.82 (dd, J=8.34, 2.2 Hz, 1H), 6.75 (m, 3H), 6.55 (brd, J=7.53 Hz, 1H), 6.48 (m, 1H), 4.81 (s, 2H), 3.74 (s, 3H), 3.70 (s, 3H), 3.59 (s, 3H), 2.30 (t, J=7.34 Hz, 2H), 2.15 (t, J=7.22 Hz, 2H), 1.92 (q, J=7.22 Hz, 2H) ppm.

¹³C NMR (150 MHz, CDCl₃): δ=173.6, 171.9, 160.3, 159.6, 143.3, 139.1, 130.2, 129.3, 121.1, 120.6, 114.1, 113.5, 113.0, 55.3, 55.2, 52.8, 51.4, 33.24, 33.16, 20.7 ppm.

MS (ESI): calcd. for C₂₁H₂₆NO₅⁺: 372.2 [M+H]⁺
found: 372.4 [M+H]⁺.

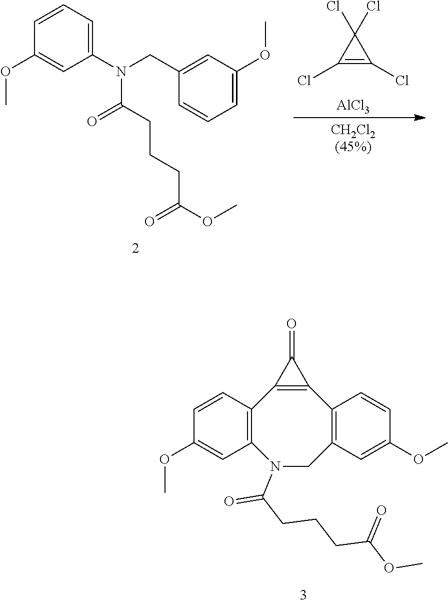

Cyclooctene 3: Tetrachlorocyclopropene (0.80 ml, 6.55 mmol) was added dropwise to a suspension of AlCl₃ (3.17 g, 23.8 mmol) in CH₂Cl₂ (50 ml) and the reaction was stirred at room temperature for 15 min. The solution was cooled to −78° C. and a solution of acylamine 2 (2.21 g, 5.95 mmol) in CH₂Cl₂ was slowly added. The reaction was allowed to warm to room temperature overnight before water (45 ml) was added and the reaction was stirred at room temperature for 30 min. The mixture was extracted with CH₂Cl₂, dried over MgSO₄ and concentrated under reduced pressure. Purification of the resultant residue by flash column chromatography (EtOAc/MeOH, 100:1, R_{f}=0.4) gave cyclooctene 3 (1.13 g, 2.67 mmol, 45%) as a yellow oil.

R_{f}=0.4 [EtOAc/MeOH, 100:1].

¹H NMR (400 MHZ, CDCl₃) δ=8.02 (d, J=8.83 Hz, 1H), 7.90 (d, J=8.51 Hz, 1H), 7.26, (d, J=2.52 Hz, 1H), 7.06 (dd, J=8.51, 2.52 Hz, 1H), 6.96 (dd, J=8.35, 2.68 Hz, 1H), 6.89 (d, J=2.52 Hz, 1H), 5.18 (d, J=14.5 hz, 1H), 4.10 (d, J=14.2 Hz, 1H), 3.93 (s, 3H), 3.92 (s, 3H), 3.56 (s, 3H), 2.33 (m, 1H), 2.14 (m, 1H), 2.00 (m, 1H), 1.93 (m, 1H), 1.75 (m, 2H) ppm.

¹³C NMR (150 MHz, CDCl₃): δ=173.2, 172.6, 163.1, 162.7, 152.5, 146.0, 143.2, 141.6, 139.0, 135.8, 135.2, 118.2, 115.5, 115.3, 114.9, 113.9, 113.7, 56.1, 55.9, 55.6, 51.5, 33.5, 32.6, 20.6 ppm.

MS (ESI): calcd. for C₂₄H₂₄NO₆⁺: 422.2 [M+H]⁺
found: 422.4 [M+H]⁺.

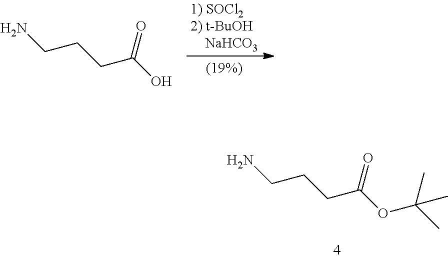

Amine 4: A solution of 4-aminobutyric acid (5.00 g, 48.5 mmol) in SOCl₂ (35 ml, 485 mmol) was stirred at room temperature for 2 h and concentrated under reduced pressure. NaHCO₃ (8.95 g, 107 mmol) and t-BuOH (105 ml) were added and the resulting suspension was stirred at room temperature overnight. All volatiles were removed under reduced pressure and the residue was portioned between EtOAc and 1M NaOH. The organic phase was washed with water and brine, dried over MgSO₄ and concentrated under reduced pressure to give amine 4 (1.50 g, 9.39 mmol, 19%) as a pale brown oil.

¹H NMR (400 MHZ, CDCl₃) δ=2.72 (t, J=7.03 Hz, 2H), 2.27 (t, J=7.47 Hz, 2H), 1.73 (q, J=7.22 Hz, 2H), 1.45 (s, 3H), 1.26 (brs, 2H) ppm.

¹³C NMR (150 MHz, CDCl₃): δ=172.9, 80.2, 41.6, 33.0, 29.1, 28.1 ppm.

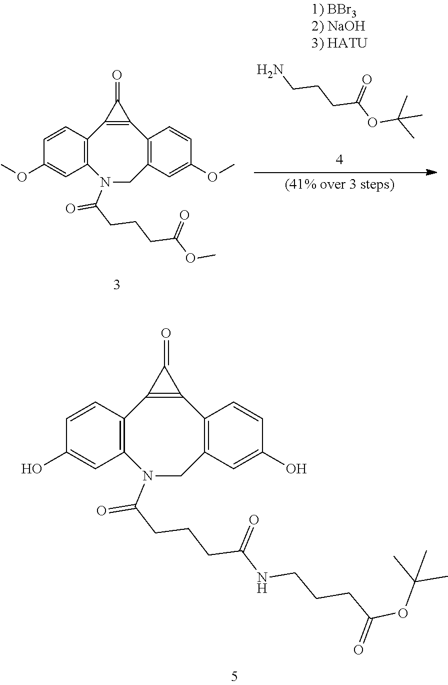

Amide 5: BBr3 (1.0M in CH₂Cl₂, 26.6 ml, 26.6 mmol) was added slowly to a solution of cyclooctane 3 (1.12 g, 2.66 mmol) in CH₂Cl₂ (128 ml) and the solution was stirred at −78° C. for 1 h and at room temperature for 48 h. The reaction was quenched with water, basified with 4M NaOH and washed with CH₂Cl₂. (Two equal batches were combined at this stage.) The aqueous phase was acidified with conc. HCl and the resultant precipitate was collected. The aqueous phase was extracted with EtOAc and the combined organic phases were dried over MgSO₄ and concentrated under reduced pressure. The solids were combined, dissolved in MeOH (13 ml), THF (13 ml) and 1M NaOH (21 ml) and stirred at room temperature for 3 h. The reaction mixture was acidified with conc. HCl and the resultant precipitate was collected. The aqueous phase was extracted with EtOAc and the combined organic phases were dried over MgSO₄ and concentrated under reduced pressure. The combined solids (1.06 g) and amine 4 (852 mg, 5.35 mmol) were dissolved in DMF (287 ml). HATU (2.03 g, 5.35 mmol and DIPEA (3.73 ml, 21.4 mmol) were sequentially added and the resulting mixture was stirred at room temperature for 20 h and concentrated under reduced pressure. Purification of the resultant residue by flash column chromatography (6% MeOH in CH₂Cl₂) gave amide 5 (1.14 g, 2.18 mmol, 41% over 3 steps) as a pale brown solid.

R_{f}=0.3 [MeOH/CH₂Cl₂, 6:94].

¹H NMR (400 MHZ, d₄-MeOH) δ8=7.91 (d, J=8.51 Hz, 1H), 7.77 (d, J=8.2 Hz, 1H), 7.14 (d, J=2.21 Hz, 1H), 7.01 (dd, J=8.51, 2.21 Hz, 1H), 6.93 (d, J=2.21 Hz, 1H), 6.87 (dd, J=8.35, 2.36 Hz, 1H), 5.10 (d, J=14.82 Hz, 1H), 4.21 (d, J=14.5 Hz, 1H), 3.06 (m, 2H), 2.31 (m, 1H), 2.18 (t, J=7.41 Hz, 2H), 1.93 (m, 3H), 1.65 (m, 4H), 1.43 (s, 9H) ppm.

¹³C NMR (150 MHz, CDCl₃): δ=175.3, 175.0, 174.4, 164.1, 163.3, 154.6, 148.0, 144.4, 142.5, 138.9, 137.0, 136.4, 120.9, 117.8, 117.4, 116.5, 115.1, 114.5, 81.7, 57.2, 39.7, 35.9, 35.0, 33.8, 28.5, 26.0, 22.8 ppm.

MS (ESI): calcd. for C₂₉H₃₃N₂O₇⁺: 521.2 [M+H]⁺
found: 521.3 [M+H]⁺.

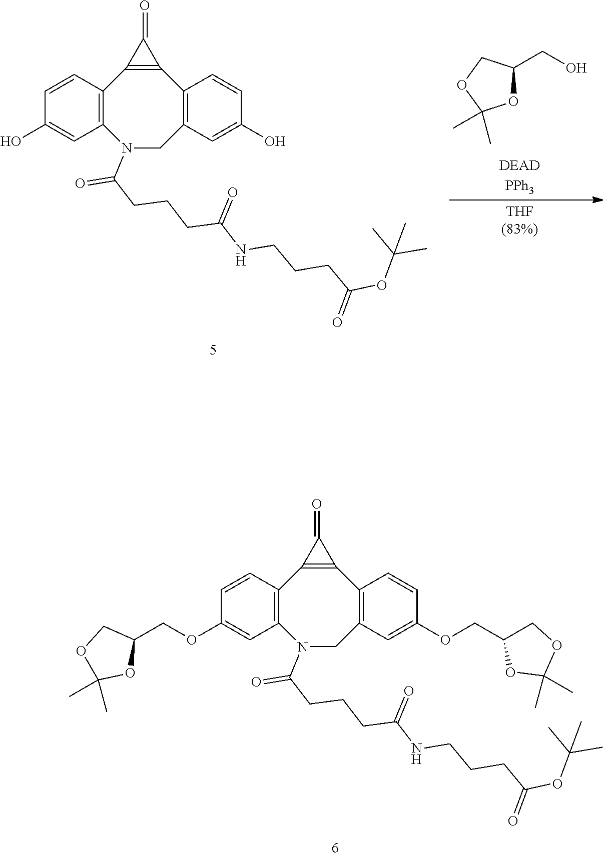

Acetonide 6: DEAD (40% in PhMe, 1.75 ml, 3.08 mmol) was added dropwise to a stirred solution of amide 5 (400 mg, 0.768 mmol), PPh₃ (808 mg, 3.08 mmol) and (R)-(−)-2,2-Dimethyl-1,3-dioxolane-4-methanol (0.38 ml, 3.08 mmol) in THF (20 ml) before the reaction was stirred at room temperature for 20 h and concentrated under reduced pressure. Purification of the resultant residue by flash column chromatography (30-60% acetone in CH₂Cl₂) gave acetonide 6 (478 mg, 0.638 mmol, 83%) as a pale white solid.

R_{f}=0.5 [acetone/CH₂Cl₂, 1:1].

¹H NMR (400 MHz, CDCl₃) δ=7.98 (d, J=8.71 Hz, 1H), 7.87 (d, J=8.31 Hz, 1H), 7.25 (m, 1H), 7.06 (d, J=8.71 Hz, 1H), 6.96 (m, 2H), 5.96 (brs, 1H), 5.12 (d, J=14.25 Hz, 1H), 4.50 (m, 2H), 4.14 (m, 6H), 4.05 (d, J=15.04 Hz, 1H), 3.92 (m, 2H), 3.16 (m, 2H), 2.33 (m, 1H), 2.20 (t, Hz=7.12 Hz, 2H), 1.99 (m, 1H), 1.91 (m, 2H), 1.70 (m, 4H), 1.45 (s, 6H), 1.40 (m, 15H) ppm.

¹³C NMR (150 MHz, CDCl₃): δ=172.8, 172.7, 172.4, 162.1, 161.6, 152.4, 145.8, 143.3, 141.6, 139.3, 135.8, 135.2, 118.8, 115.8, 115.2, 114.7, 114.2, 110.1, 109.9, 80.6, 79.77, 73.74, 73.70, 69.4, 69.0, 66.6, 66.5, 56.1, 39.1, 34.8, 33.5, 32.9, 30.9, 29.3, 28.1, 26.8, 25.32, 25.27, 24.6 ppm.

MS (ESI): calcd. for C₄₁H₅₃N₂O₁₁⁺: 749.4 [M+H]⁺
found: 749.4 [M+H]⁺.

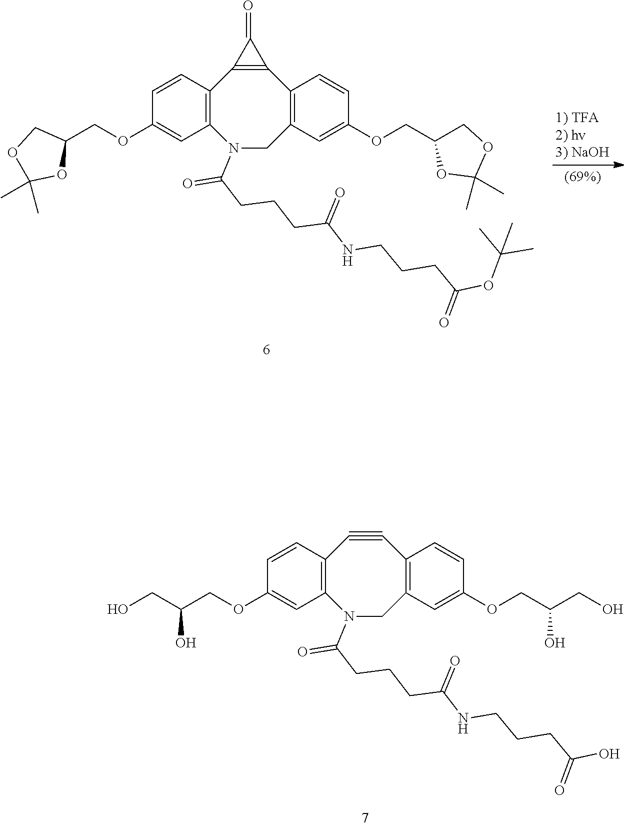 [SMILES: CC(C)(C)OC(=O)CCCNC(=O)CCCC(=O)N1Cc2cc(OC[C@@H]3COC(C)(C)O3)ccc2-c2c(c2=O)-c2ccc(OC[C@@H]3COC(C)(C)O3)cc21 ; O=C(O)CCCNC(=O)CCCC(=O)N1Cc2cc(OC[C@@H](O)CO)ccc2C#Cc2ccc(OC[C@@H](O)CO)cc21]

Alkyne 7: iPr₃SiH (0.20 ml), water (0.2 ml) and TFA (6 ml) were added to a solution of acetonide 6 (400 mg, 0.534 mmol) in CH₂Cl₂ (2 ml). The reaction was stirred at room temperature for 6 h and concentrated. The residue was dissolved in MeOH (20 ml), DIPEA (0.94 ml) was added and the reaction was irradiated (360 nm) for 2 h and concentrated. The residue was dissolved in MeOH (3 ml) and 1M NaOH (2 ml) and stirred at room temperature for 1 h. The reaction mixture was directly submitted to reversed phase HPLC chromatography (YMC-Triart C18, 29-45% MeCN in H₂O, 0.1% TFA, over 30 min) to give alkyne 7 (215 mg, 0.368 mmol, 69% over 3 steps) as a white solid.

¹H NMR (400 MHZ, d₄-MeOH) δ=7.29 (d, J=8.39 Hz, 1H), 7.27. (d, J=2.29 Hz, 1H), 7.12 (m, 2H), 7.03 (dd, J=8.56, 2.48 Hz, 1H), 6.90 (dd, J=8.39, 2.29 Hz, 1H), 5.04 (d, J=13.73 Hz, 1H), 4.13 (m, 2H), 4.04 (m, 2H), 3.99 (m, 2H), 3.68 (m, 5H), 3.09 (t, J=7.06 Hz, 2H), 2.31 (m, 1H), 2.25 (t, J=7.44 Hz, 2H), 1.95 (m, 3H), 1.68 (m, 4H) ppm.

¹³C NMR (150 MHz, d₄-MeOH): δ=177.0, 175.4, 175.1, 160.7, 160.3, 153.8, 151.1, 128.5, 127.3, 120.51, 120.45, 117.7, 116.9, 116.2, 115.9, 115.0, 114.7, 107.9, 71.9, 71.8, 71.4, 70.7, 64.3, 64.2, 57.0, 39.8, 36.1, 35.2, 32.4, 25.9, 22.9 ppm.

MS (ESI): calcd. for C₃₀H₃₇N₂O₁₀⁺: 585.2 [M+H]⁺
found: 585.4 [M+H]⁺.

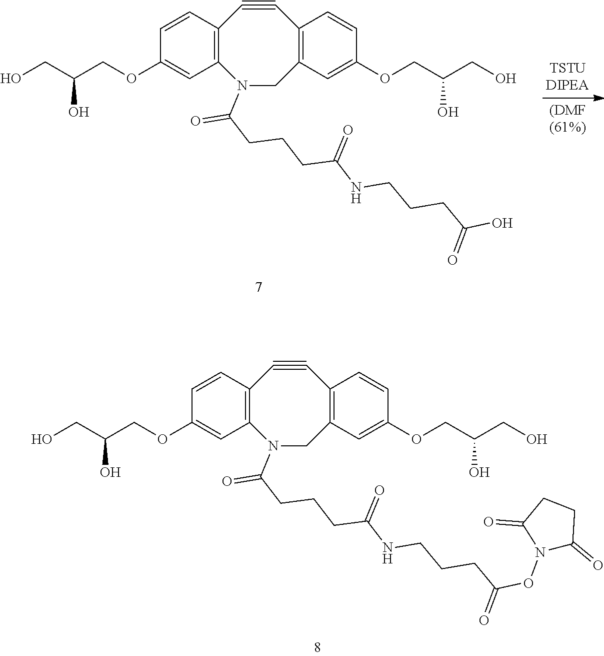 [SMILES: O=C(O)CCCNC(=O)CCCC(=O)N1Cc2cc(OC[C@@H](O)CO)ccc2C#Cc2ccc(OC[C@@H](O)CO)cc21 ; O=C(CCCC(=O)N1Cc2cc(OC[C@@H](O)CO)ccc2C#Cc2ccc(OC[C@@H](O)CO)cc21)NCCCC(=O)ON1C(=O)CCC1=O]

NHS-Ester 8: DIPEA (31 μL, 0.176 mmol) was added to a solution of alkyne 7 (43 mg, 0.074 mmol) and TSTU (44 mg, 0.147 mmol) in DMF (2 ml) and the solution was stirred at room temperature for 2 h. Concentration of the reaction under reduced pressure and purification of the resultant residue by reversed phase HPLC chromatohraphy (YMC-Triart C18, 32-48% MeCN in H₂O, 0.1% TFA, over 30 min) gave NHS-ester 8 (31 mg, 0.045 mmol, 61%) as a white solid.

¹H NMR (400 MHZ, d₄-MeOH) δ=7.30 (d, J=8.53 Hz, 1H), 7.27 (d, J=2.38 Hz, 1H), 7.13 (m, 2H), 7.03 (dd, J=8.53, 2.51 Hz, 1H), 6.90 (dd, J=8.41, 2.51 Hz, 1H), 5.04 (d, J=13.93 Hz, 1H), 4.11 (m, 2H), 4.00 (m, 4H), 3.70 (d, J=9.16 Hz, 1H), 3.66 (m, 4H), 3.16 (t, J=, 6.84 Hz, 2H), 2.80 (s, 4H), 2.60 (t, J=7.4 Hz, 2H), 2.27 (m, 1H), 1.93 (m, 3H), 1.79 (q, J=7.12 Hz, 2H), 1.65 (m, 2H) ppm.

¹³C NMR (150 MHz, d₄-MeOH): δ=174.0, 173.5, 170.4, 168.5, 159.1, 158.7, 155.9, 152.2, 149.5, 127.3, 127.0, 125.7, 119.5, 118.9, 116.1, 115.3, 114.6, 114.3, 70.3, 70.2, 69.5, 62.7, 62.6, 55.4, 37.8, 34.5, 33.6, 27.7, 25.1, 24.0, 21.3 ppm.

MS (ESI): calcd. for C₃₄H₄₀N₃O₁₂⁺: 682.3 [M+H]⁺
found: 682.4 [M+H]⁺.

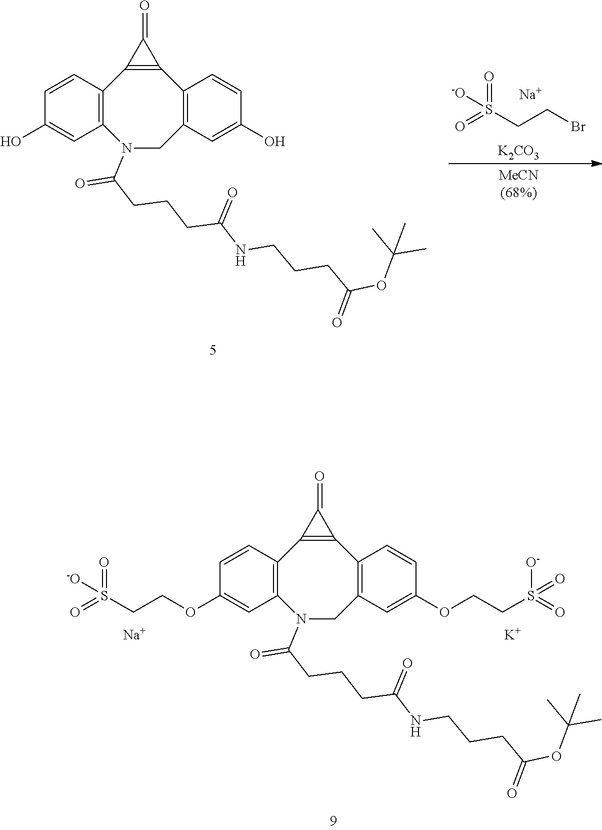

Sulfonic acid 9: A suspension of amide 5 (100 mg, 0.192 mmol), K₂CO₃ (159 mg, 1.15 mmol) and sodium 2-bromoethanesulfonate (243 mg, 1.15 mmol) in MeCN (2.8 ml) was stirred at 80° C. for 5 d. Purification of the reaction mixture by reversed phase HPLC chromatography (YMC-Triart C18, 23-39% MeCN in H₂O, 0.1% TFA, over 30 min) gave sulfonic acid 9 (104 mg, 0.131 mmol, 68%) as a white solid.

¹H NMR (400 MHZ, d₄-MeOH) δ=7.92 (d, J=8.51 Hz, 1H), 7.76 (d, J=8.51 Hz, 1H), 7.26 (d, J=1.89 Hz, 1H), 7.19 (d, J=2.21 Hz, 1H), 7.14 (dd, J=8.51, 2.21 Hz, 1H), 6.98 (dd, J=8.51, 1.89 Hz, 1H), 5.10 (d, J=14.5 Hz, 1H), 4.43 (m, 4H), 4.13 (d, J=14.5 Hz, 1H), 3.24 (m, 5H), 3.10 (t, J=6.94 Hz, 2H), 2.38 (m, 1H), 2.21 (t, J=7.25 Hz, 1H), 2.12 (m, 1H), 2.07 (m, 1H), 1.84 (m, 1H), 1.62 (m, 4H), 1.33 (s, 5H), 1.33 (s, 5.5H) 1.13 (s, 2H9, 1.09 (s, 0.5H) ppm (mixture of rotamers).

¹³C NMR (150 MHz, d₄-MeOH): δ=177.0, 176.9, 174.8, 174.1, 173.7, 163.9, 163.2, 154.3, 147.4, 144.0, 143.0, 139.6, 136.6, 136.1, 119.9, 117.0, 116.3, 115.6, 115.2, 81.5, 65.7, 65.2, 56.9, 51.5, 51.3, 40.7, 40.6, 36.7, 34.63, 34.55, 33.2, 31.7, 31.0, 28.2, 27.1, 24.9, 24.8, 22.43, 22.39 ppm (mixture of rotamers).

MS (ESI): calcd. for C₃₃H₄₁N₂O₁₃S₂⁺: 737.2 [M+H]⁺
found: 737.4 [M+H]⁺.

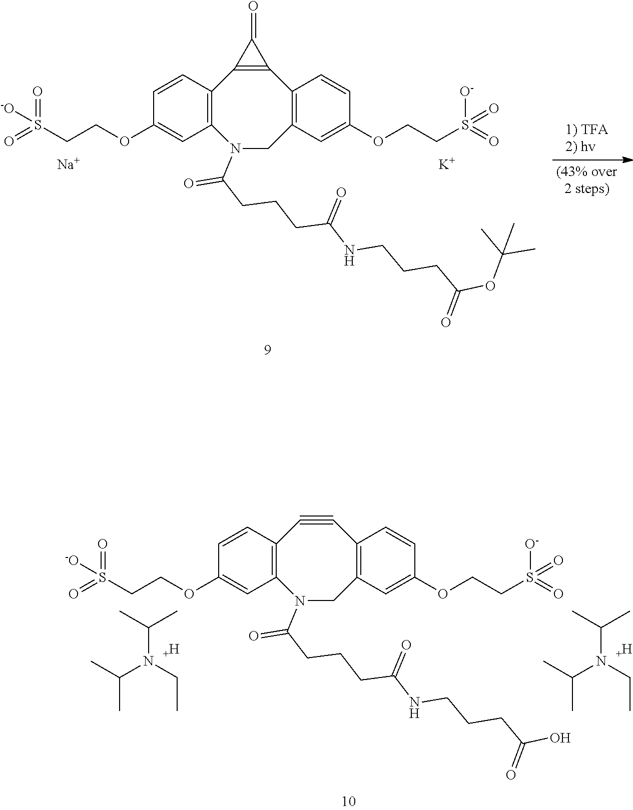

Carboxylic acid 10: : A solution of sulfonic acid 9 (84 mg, 0.105 mmol) in CH₂Cl₂ (0.83 ml), iPr₃SiH (0.08 ml), water (0.08 ml) and TFA (2.5 ml) was stirred at room temperature for 2 h and concentrated under reduced pressure. The residue was co-evaporated two times with acetone and MeCN. The resultant residue was dissolved in MeOH (6.7 ml) and DIPEA (0.40 ml), irradiated (360 nm) for 1.5 h and concentrated under reduced pressure. Purification of the resultant residue by reversed phase HPLC chromatography (YMC-Triart C18, 22-38% MeCN in H₂O, 0.1% TFA, over 30 min) gave carboxylic acid 10 (41 mg, 0.045 mmol, 43% over 2 steps) as a white solid.

¹H NMR (400 MHZ, d₄-MeOH) δ7.29 (d, J=8.53 Hz, 1H), 7.25 (d, J=2.26 Hz, 1H), 7.15 (d, J=2.38 Hz, 1H), 7.12 (d, J=8.41 Hz, 1H)), 7.03 (dd, J=8.60, 2.45 Hz, 1H), 6.90 (dd, J=8.53, 2.38 Hz, 1H), 5.04 (d, J=14.05 Hz, 1H), 4.42 (m, 5H), 3.69 (spt, J=6.61 Hz, 4H), 3.29 (m, 4H), 3.18 (m, 6H), 2.30 (m, 4H), 2.07 (m, 1H), 1.90 (m 1H), 1.73 (m, 4H), 1.34 (m, 30H) ppm.

¹³C NMR (150 MHz, d₄-MeOH): δ=174.9, 173.7, 173.1, 158.6, 158.2, 152.1, 149.6, 127.0, 125.8, 119.1, 116.3, 115.4, 114.7, 114.5, 113.2, 113.0, 106.4, 64.0, 63.6, 55.4, 50.4, 50.1, 42.4, 38.8, 34.0, 33.5, 30.5, 23.8, 21.3, 17.3, 15.9 ppm.

MS (ESI): calcd. for C₂₈H₃₃N₂O₁₂S₂⁺: 653.1[M+H]⁺
found: 653.1[M+H]⁺.

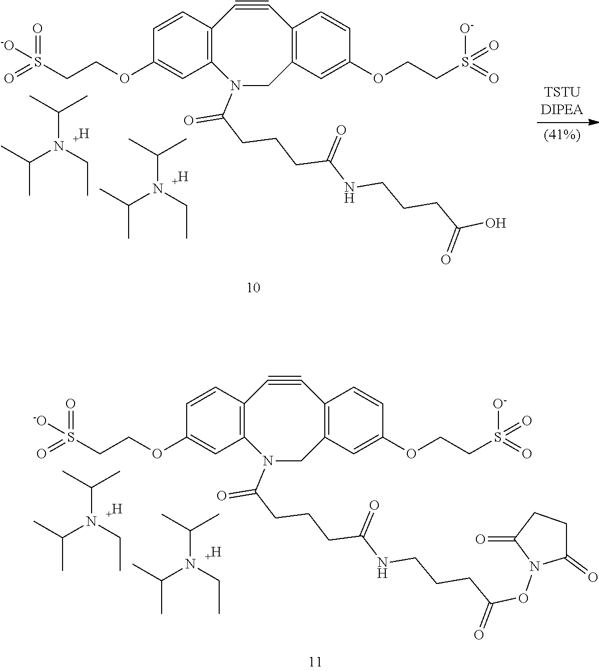

NHS-Ester 11: DIPEA (0.17 ml, 0.998 mmol) was added to a solution of carboxylic acid 10 (87 mg, 0.095 mmol) and TSTU (80 mg, 0.266 mmol) in DMF (3 ml). The reaction was stirred at room temperature for 2 h and concentrated under reduced pressure. Purification of the resultant residue by reversed phase HPLC chromatography (YMC-Triart C18, 24-40% MeCN in H₂O, 0.1% TFA, over 30 min) NHS-ester 10 (39 mg, 0.045 mmol, 41) as a white solid.

¹H NMR (400 MHZ, d₄-MeOH) δ=7.31 (d, J=8.53 Hz, 1H), 7.27 (d, J=2.51 Hz, 1H), 7.17 (d, J=2.51 Hz, 1H), 7.14 (d, J=8.41 Hz, 1H), 7.05 (dd, J=8.53, 2.51 Hz, 1H), 6.92 (dd, J=8.47, 2.45 Hz, 1H), 5.06 (d, J=13.93 Hz, 1H), 4.44 (m, 5H), 3.71 (m, 4H), 3.31 (m, 4H), 3.21 (m, 6H), 2.83 (s, 4H), 2.68 (m, 1H), 2.63 (t, J=7.34 Hz, 1H), 2.34 (m, 2H), 2.26 (q, J=7.49 Hz, 1H), 2.08 (m, 1H), 1.90 (m, 4H), 1.36 (m, 30H) ppm.

MS (ESI): calcd. for C₃₂H₃₆N₃O ₁₄S₂⁺: 750.2 [M+H]⁺
found: 750.4 [M+H]⁺.

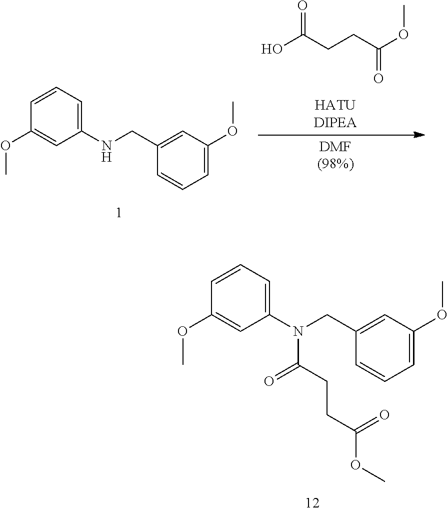

Acyl amine 12: DIPEA (3.85 ml, 11.1 mmol) was added to a solution of aniline 1 (5.66 g, 11.1 mmol), mono methyl succinate (1.98 g, 14.9 mmol) and HATU (5.66 g, 14.9 mmol) in DMF (28 ml) and the reaction was allowed to stir at room temperature for 3 d. The mixture was diluted with EtOAc, washed with 1M HCl, water and brine, dried over MgSO₄ and concentrated under reduced pressure. Purification of the resultant residue by flash column chromatography (EtOAc:hexane=1:1, R_{f}=0.5) gave acylamine 12 (3.85 g, 10.8 mmol, 98%) as a pale yellow oil.

R_{f}=0.5 [hexane/EtOAc, 1:1].

¹H NMR (400 MHZ, CDCl₃) δ=7.22 (t, J=8.09 Hz, 1H), 7.16 (dd, J=9.06 Hz, 7.28 Hz, 1H), 6.83 (dd, J=8.28, 2.13 Hz, 1H), 6.76 (m, 3H), 6.64 (d, J=7.65 Hz, 1H), 6.56 (m, 1H), 4.86 (s, 2H), 3.75 (s, 1H), 3.71 (s, 1H), 3.66 (s, 1H), 2.63 (m, 2H), 2.39 (m, 2H) ppm.

¹³C NMR (150 MHz, CDCl₃): δ=173.5, 171.2, 160.3, 159.6, 143.2, 139.1, 130.2, 129.3, 121.0, 120.6, 114.1, 113.9, 113.1, 55.3, 55.2, 53.0, 51.7, 29.3 ppm.

MS (ESI): calcd. for C₂₀H₂₄NO₅⁺: 358.2[M+H]⁺
found: 358.4 [M+H]⁺.

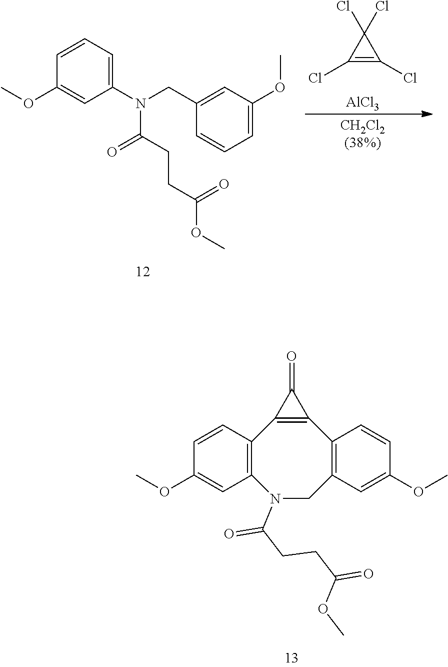

Cyclooctene 13: Tetrachlorocyclopropene (0.57 ml, 5.72 mmol) was added dropwise to a suspension of AlCl₃ (2.74 g, 20.7 mmol) in CH₂Cl₂ (45 ml) and the reaction was stirred at room temperature for 15 min. The solution was cooled to —78° C. and a solution of acylamine 12 (1.85 g, 5.16 mmol) in CH₂Cl₂ (30 ml) was slowly added. The reaction was allowed to warm to room temperature overnight before water (39 ml) was added and the reaction was stirred at room temperature for 30 min. The mixture was extracted with CH₂Cl₂, dried over MgSO₄ and concentrated under reduced pressure. Purification of the resultant residue by flash column chromatography (EtOAc/MeOH, 100:1, R_{f}=0.4) gave cyclooctene 13 (1.60 g, 3.93 mmol, 38%) as a yellowish oil.

R_{f}=0.4 [EtOAc/MeOH, 100:1].

¹H NMR (400 MHZ, CDC₃) δ8.00 (d, J=8.66 Hz, 1H), 7.90 (d, J=8.53 Hz, 1H), 7.25 (d, J=2.51 Hz, 1H), 7.18 (d, J=2.51 Hz, 1H), 7.06 (dd, J=8.66, 2.51 Hz, 1H), 6.95 (dd, J=8.47, 2.57 Hz, 1H), 5.21 (d, J=14.4 Hz, 1H), 4.12 (d, J=14.4 Hz, 1H), 3.94 (s, 3H), 3.91 (s, 3H), 3.59 (s, 3H), 2.76 (m, 1H), 2.66 (m, 1H), 2.36 (m, 1H), 1.94 (m, 1H) ppm.

MS (ESI): calcd. for C₂₄H₂₄NO₆⁺: 408.1 [M+H]⁺
found: 408.3 [M+H]⁺.

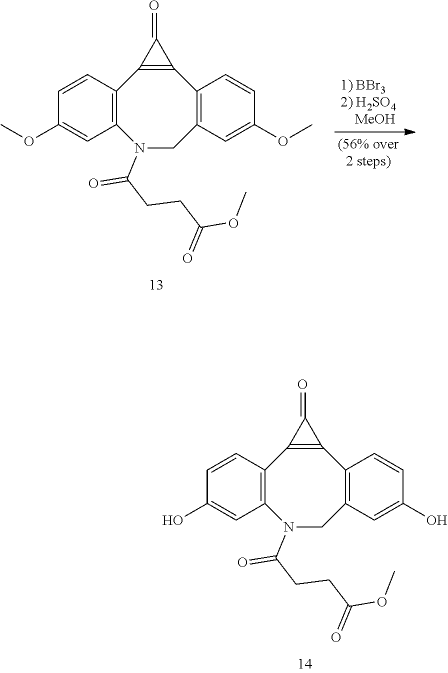

Phenol 14: BBr₃ (1.0M in CH₂Cl₂, 20 ml, 20 mmol) was added slowly to a solution of cyclooctene 13 (817 mg, 2.00 mmol) in CH₂Cl₂ (95 ml) and the solution was stirred at —78° C. for 1 h and at room temperature for 48 h. The reaction was quenched with water, basified with 4M NaOH and washed with CH₂Cl₂. The aqueous phase was acidified with conc. HCl and the resultant precipitate was collected. The aqueous phase was extracted with EtOAc and the combined organic phases were dried over MgSO₄ and concentrated under reduced pressure. The combined solids were dissolved in MeOH (20 ml), conc. H₂SO₄ (0.4 ml) was added and the reaction was stirred at 65° C. for 3 h. The mixture was cooled to room temperature, diluted with water and extracted with EtOAc. The combined organic phases were dried over MgSO₄ and concentrated under reduced pressure. Purification of the resulting residue by flash column chromatography (6-8% MeOH in CH₂Cl₂) gave phenol 14 (424 mg, 1.12 mmol, 56% over 2 steps) as a brownish solid.

MS (ESI): calcd. for C₂₁H₁₈NO₆⁺: 380.1 [M+H]⁺
found: 380.3 [M+H]⁺.

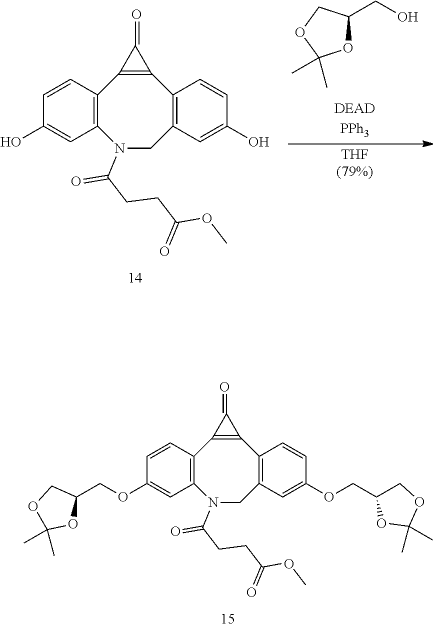

Acetonide 15: DEAD (40% in PhMe, 94 μL, 0.207 mmol) was added dropwise to a stirred solution of phenol 14 (26 mg, 0.069 mmol), PPh₃ (54 mg, 0.207 mmol) and (R)-(−)-2,2-dimethyl-1,3-dioxolane-4-methanol (26 μL, 0.207 mmol) in THF (1.5 ml) before the reaction was stirred at room temperature for 20 h and concentrated under reduced pressure. Purification of the resultant residue by flash column chromatography (10-30% acetone in CH₂Cl₂, R_{f}=0.3 (20% acetone in CH₂Cl₂)) gave acetonide 15 (33 mg, 0.054 mmol, 79%) as a pale white solid.

R_{f}=0.3 [acetone/CH₂Cl_{2, 1:4].}

¹H NMR (400 MHZ, CDCl₃) δ=7.99 (d, J=8.66 Hz, 1H), 7.89 (d, J=8.41 Hz, 1H), 7.25 (t, J=2.64 Hz, 1H), 7.21 (t, J=2.26 Hz, 1H), 7.07 (m, 1H), 8.96 (dt, J=8.47, 2.60 Hz, 1H), 5.17 (d, J=14.3 Hz, 1H), 4.50 (m, 2H), 4.18 (m, 7H), 3.93 (m, 2H), 3.58 (s, 3H), 2.68 (m, 2H), 2.35 (m, 1H), 1.92 (m, 1H), 1.47 (s, 6H), 1.41 (s, 6H) ppm.

MS (ESI): calcd. for C₃₃H₃₈NO₁₀⁺: 608.2 [M+H]⁺
found: 608.3 [M+H]⁺.

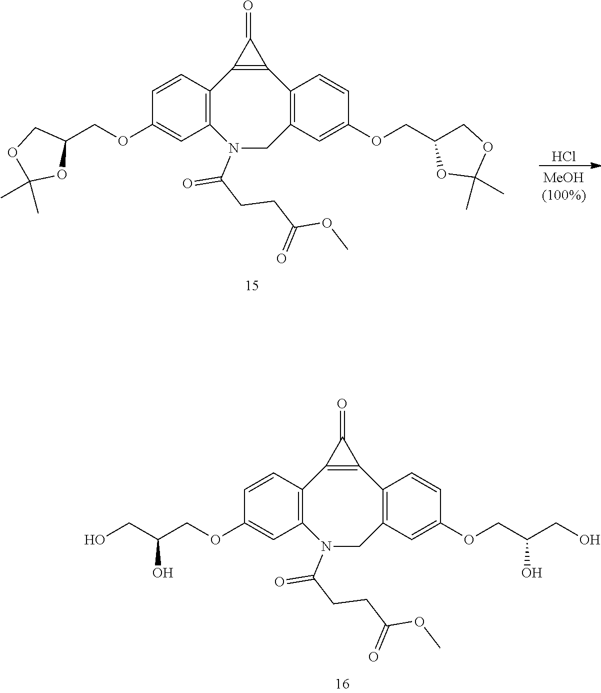

Tetraol 16: 1M HCl (0.1 ml) was added slowly to a solution of acetonide 15 (11 mg, 0.018 mmol) in MeOH (0.2 ml) and the reaction was stirred at room temperature for 2 h. The mixture was directly submitted to flash column chromatography (10% MeOH in CH₂Cl₂) to give tetraol 16 (9.7 mg, 0.018 mmol, 100%) as a colorless oil.

R_{f}=0.2 [methanol/CH₂Cl₂, 1:9].

¹H NMR (400 MHZ, d₄-MeOH) δ=8.04 (d, J=8.66 Hz, 1H), 7.88 (d, J=8.53 Hz, 1H), 7.34 (m, 2H), 7.24 (d, J=8.60, 2.45 Hz, 1H), 7.08 (dd, J=8.53, 2.51 Hz, 1H), 5.17 (d, J=14.6 Hz, 1H), 4.28 (d, J=14.3 Hz, 1H), 4.20 (m, 4H), 4.02 (m, 2H), 3.70 (m, 4H), 3.46 (s, 3H). 2.65 (m, 1H), 2.42 (m, 2H), 2.03 (m, 1H) ppm.

MS (ESI): calcd. for C₂₇H₃₀NO₁₀⁺: 528.2 [M+H]⁺
found: 528.4 [M+H]⁺.

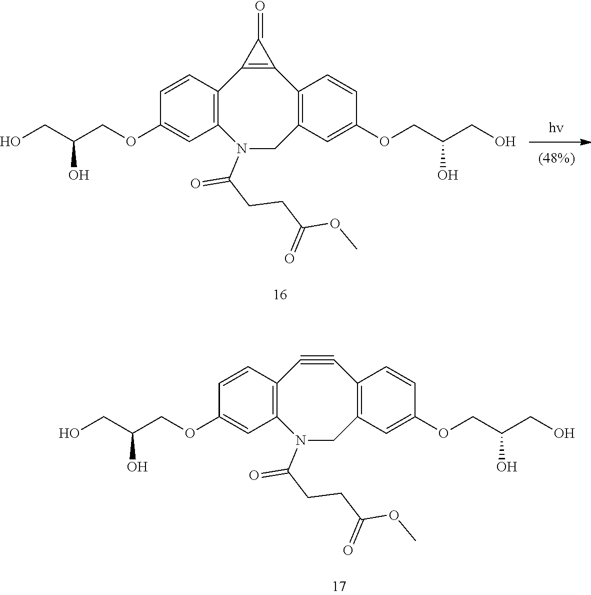 [SMILES: COC(=O)CCC(=O)N1Cc2cc(OC[C@@H](O)CO)ccc2-c2c(c2=O)-c2ccc(OC[C@@H](O)CO)cc21 ; COC(=O)CCC(=O)N1Cc2cc(OC[C@@H](O)CO)ccc2C#Cc2ccc(OC[C@@H](O)CO)cc21]

Alkyne 17: A solution of tetraol 16 (9.7 mg, 0.018 mmol) in MeOH (1.5 ml) was irradiated (360 nm) for 30 min. Concentration of the solution under reduced pressure and purification of the resulting residue by flash column chromatography (10% MeOH in CH₂Cl₂, R_{f}=0.2) gave alkyne 17 (4.3 mg, 8.6 μmol, 48%) as a colorless oil.

R_{f}=0.2 [methanol/CH₂Cl₂, 1:9].

¹H NMR (400 MHZ, d₄-MeOH) δ=7.31 (d, J=8.53 Hz, 1H), 7.26 (m, 2H), 7.12 (d, J=8.41 Hz, 1H), 7.05 (dd, J=8.6, 2.57 Hz, 1H), 6.90 (dd, J=8.47, 2.57 Hz, 1H) 5.014 (d, J=14.1 Hz, 1H)., 4.06 (m, 6H), 3.73 (d, J=13.8 Hz, 1H), 3.67 (m, 4H), 3.53 (s, 3H), 2.74 (m, 1H), 2.47 (m, 1H), 2.38 (m, 1H), 2.01 (m, 1H) ppm.

MS (ESI): calcd. for C₃₃H₃₈NO₁₀⁺: 500.2 [M+H]⁺
found: 500.4 [M+H]⁺.

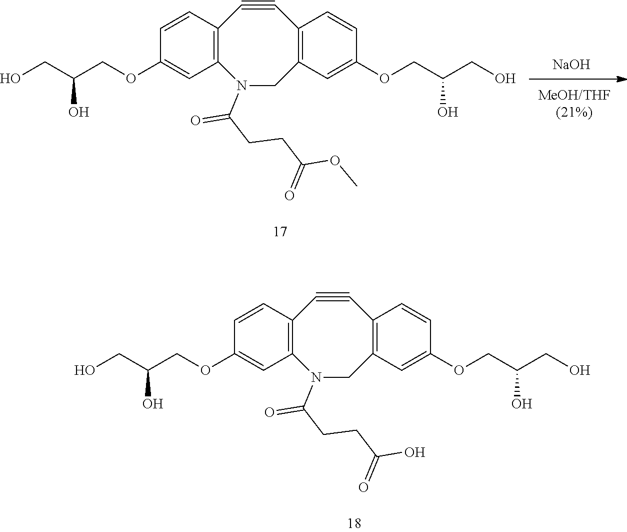

Carboxylic acid 18: 1M NaOH (100 μL) was added to a solution of alkyne 17 (15.8 mg, 0.032 mmol) in THF (1 ml) and MeOH (1 ml) and the reaction was stirred at room temperature overnight. Concentration of the reaction under reduced pressure and purification of the resulting residue by reversed phase HPLC chromatography (Chromolith RP18e, C₁₈, 18-34% MeCN in H₂O, 0.1% TFA, over 30 min) gave carboxylic acid 18 (3.2 mg, 6.6 μmol, 21%) as a white solid.

¹H NMR (400 MHZ, d₄-MeOH) δ=7.30 (m, 2H), 7.26 (d, J=2.38 Hz, 1H), 7.12 (d, J=8.41 Hz, 1H), 7.04 (dd, J=8.53, 2.51 Hz, 1H), 6.90 (dd, J=8.41, 2.51 ppm.

MS (ESI): calcd. for C₂₅H₂₈NO₉⁺: 486.2 [M+H]⁺
found: 486.4 [M+H]⁺.

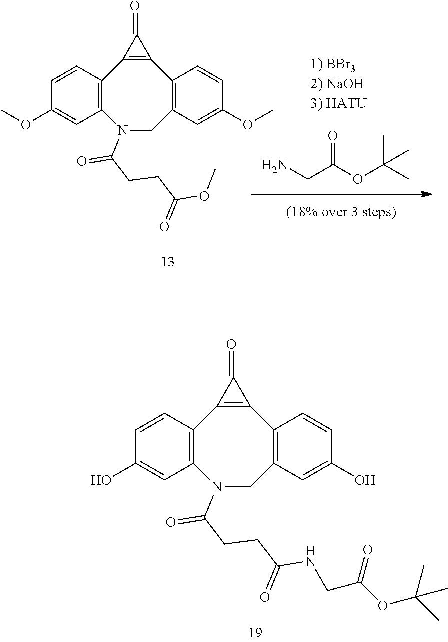

tert-Butylester 19: BBr₃ (1.0M in CH₂Cl₂, 39ml, 39 mmol) was added slowly to a solution of cyclooctane 13 (1.60 g, 3.93 mmol) in CH₂Cl₂ (190 ml) and the solution was stirred at −78° C. for 1 h and at room temperature for 48 h. The reaction was quenched with water, basified with 4M NaOH and washed with CH₂Cl₂. The aqueous phase was acidified with conc. HCl and the resultant precipitate was collected. The aqueous phase was extracted with EtOAc and the combined organic phases were dried over MgSO₄ and concentrated under reduced pressure. The solids were combined, dissolved in MeOH (10 ml), THF (10 ml) and 1M NaOH (16 ml) and stirred at room temperature for 3 h. The reaction mixture was acidified with conc. HCl and the resultant precipitate was collected. The aqueous phase was extracted with EtOAc and the combined organic phases were dried over MgSO₄ and concentrated under reduced pressure. The combined solids (702 mg) and glycine tert-butyl ester hydrochloride (322 mg, 1.92 mmol) were dissolved in DMF (22 ml). HATU (728 mg, 1.92 mmol and DIPEA (1.32 ml, 7.63 mmol) were sequentially added and the resulting mixture was stirred at room temperature for 20 h and concentrated under reduced pressure. Purification of the resultant residue by flash column chromatography (4-6% MeOH in CH₂Cl₂, R_{f}=0.4, 6% MeOH in CH₂Cl₂) gave tert-butylester 19 (333 mg, 0.696 mmol, 18% over 3 steps) as a brownish solid.

R_{f}=0.4 [methanol/CH₂Cl₂, 6:94].

¹H NMR (400 MHZ, d₄-MeOH) δ=7.90 (d, J=8.53 Hz, 1H), 7.75 (d, J=8.28 Hz, 1H), 7,12 (m, 1H), 7.02 (m, 2H), 6.85 (dd, J=8.34, 2.32 ppm.

MS (ESI): calcd. for C₂₆H₂₇N₂O₇⁺: 479.2 [M+H]⁺
found: 479.0 [M+H]⁺.

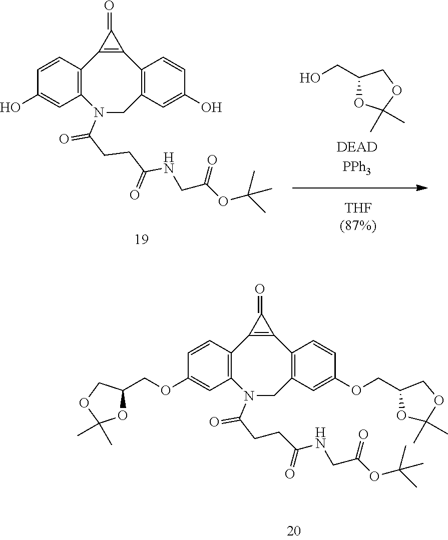

Acetonide 20: DEAD (40% in PhMe, 0.48 ml, 0.836 mmol) was added dropwise to a solution of tert-butylester 19 (100 mg, 0.209 mmol), PPh₃ (219 mg, 0.836 mmol) and (R)-(−)-2,2-dimethyl-1,3-dioxolane-4-methanol (103 μL, 0.836 mmol) in THF (5.5 ml). After stirring the reaction at room temperature overnight, the solvent was removed under reduced pressure and the residue was purified by column chromatography (30-50% acetone in CH₂Cl₂, R_{f}=0.5 (40% acetone in CH₂Cl₂)). Acetonide 20 (128 mg, 0.181 mmol, 87%) was obtained as a white solid.

R_{f}=0.5 [acetonel/CH₂Cl₂, 2:3].

¹H NMR (400 MHz, CDCl₃) δ=7.95 (d, J=8.28 Hz, 1H), 7.86 (d, J=8.41 Hz, 1H), 7.24 (m, 2H), 7.05 (dt, J=8.66, 2.89 Hz, 1H), 6.94 (dt, J=8.53, 2.32 Hz, 1H), 6.05 (brt, J=4.77 Hz, 1H), 5.15 (d, J=14.4 Hz, 1H), 4.49 (m, 2H), 4.12 (m, 7H), 3.90 (m, 2H), 3.83 (d, J=5-14 Hz, 2H), 2.85 (m, 1H), 2,60 (m, 1H), 2.16 (m, 1H), 1.89 (m, 1H), 1.45 (s, 6H), 1.44 (s, 9H), 1.39 (s, 6H) ppm.

MS (ESI): calcd. for C₃₈H₄₇N₂O₁₁⁺: 707.3 [M+H]⁺
found: 707.6 [M+H]⁺.

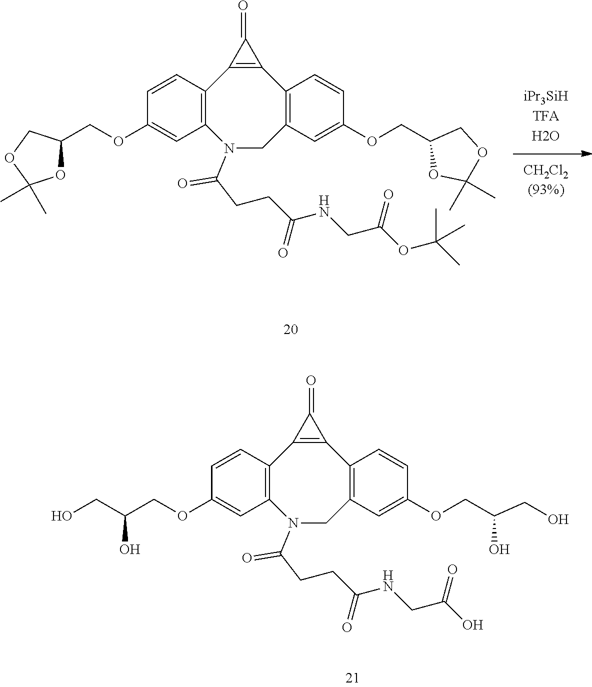

Carboxylic acid 21: TFA (0.5 ml) was added to a mixture of acetonide 20 (64 mg, 0.091 mmol), iPr₃SiH (0.1 ml), water (0.1 ml) and CH₂Cl₂ (1 ml). The reaction was stirred at room temperature for 6 h, diluted with CH₂Cl₂ and extracted with water. The combined aqueous phases were lyophilized to give carboxylic acid 21 (48.5 mg, 0.085 mmol, 93%) ¹H NMR (400 MHZ, d₄-MeOH) δ=8.00 (d, J=8.66 Hz, 1H), 7.84 (d, J=8.53 Hz, 1H), 7.36 (t, J=2.13 Hz, 1H), 7.31 (d, J=2.51 Hz, 1H), 7.20 (dd, J=8.60, 2.32 Hz, 1H), 7.04 (dd, J=8.47, 2.45 Hz, 1H), 5.15 (d, J=14.56 Hz, 1H), 4.18 (m, 5H), 4.02 (m, 2H), 3.77 (s, 2H), 3.70 (m, 4H), 2.73 (m, 1H), 2.44 (m, 1H), 2.26 (m, 1H), 2.01 (m, 1H) ppm.

¹³C NMR (150 MHz, d₄-MeOH): δ=174.8, 174.3, 173.1, 163.2, 162.3, 153.2, 146.2, 142.6, 141.6, 138.3, 135.2, 134.6, 118.5, 115.6, 115.2, 115.0, 114.2, 113.9, 70.2, 70.1, 69.9, 69.4, 62.6, 62.5, 55.7, 40.3, 30.0, 29.4 ppm.

MS (ESI): calcd. for C₃₅H₄₅N₄O₁₂⁺: 571.2 [M+H]⁺
found: 571.4 [M+H]⁺.

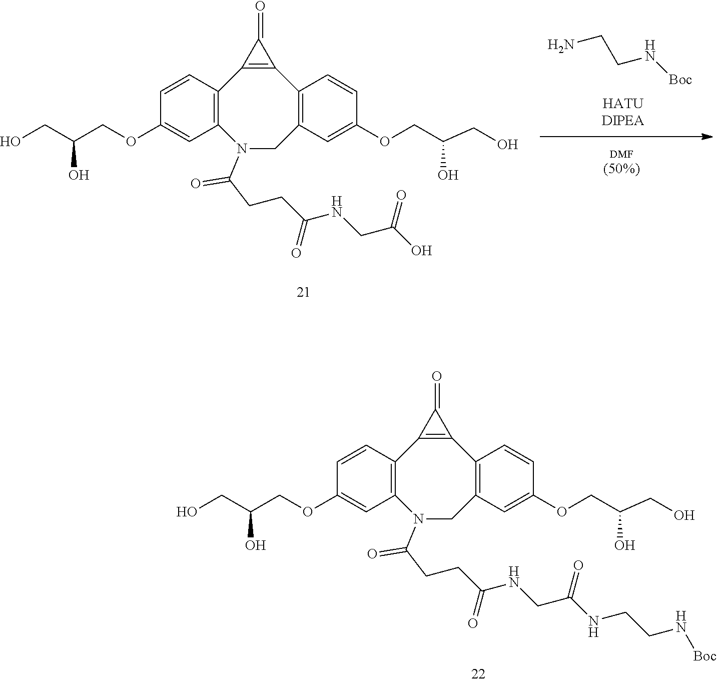 [SMILES: O=C(O)CNC(=O)CCC(=O)N1Cc2cc(OC[C@@H](O)CO)ccc2-c2c(c2=O)-c2ccc(OC[C@@H](O)CO)cc21 ; CC(C)(C)OC(=O)NCCN ; CC(C)(C)OC(=O)NCCNC(=O)CNC(=O)CCC(=O)N1Cc2cc(OC[C@@H](O)CO)ccc2-c2c(c2=O)-c2ccc(OC[C@@H](O)CO)cc21]

Amide 22: DIPEA (29 μL, 0.168 mmol) was added to a solution of carboxylic acid 21 (48 mg, 0.084 mmol), N-Boc-ethylenediamine (27 mg, 0.168 mmol) and HATU (64 mg, 0.168 mmol) In DMF (1 ml). After stirring the reaction at room temperature overnight, the solvent was removed under reduced pressure and the residue was purified by reversed phase HPLC chromatography (Chromolith RP18e, C₁₈, 14-30% MeCN in H₂O, 0.1% TFA, over 30 min). Amide 22 (30 mg, 0.042 mmol, 50%) was obtained as a white solid.

¹H NMR (400 MHZ, d₄-MeOH) δ=8.02 (d, J=8.53 Hz, 1H), 7.86 (d, J=8.53, 1H), 7.34 (m, 2H), 7.23 (dd, J=8.66, 2.38 Hz, 1H), 7.07 (dd, J=8.53, 2.51 Hz, 1H), 5.21 (d, J=14.8 Hz, 1H), 4.19 (m, 5H), 4.02 (m, 2H), 3.82 (d, J=16.8 Hz, 1H), 3.69 (m, 4H), 3.60 (d, J=16.8 Hz, 1H), 3.18 (m, 2H), 2.83 (m, 1H), 2.29 (m, 2H), 2.03 (m, 1H), 1.42 (s, 9H) ppm.

MS (ESI): calcd. for C₃₅H₄₅N₄O₁₂⁺: 713.3 [M+H]⁺
found: 713.6 [M+H]⁺.

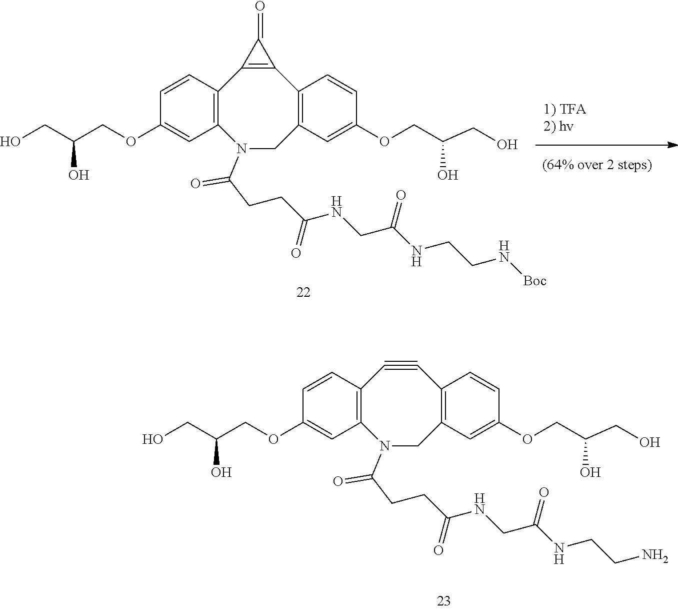 [SMILES: CC(C)(C)OC(=O)NCCNC(=O)CNC(=O)CCC(=O)N1Cc2cc(OC[C@@H](O)CO)ccc2-c2c(c2=O)-c2ccc(OC[C@@H](O)CO)cc21 ; NCCNC(=O)CNC(=O)CCC(=O)N1Cc2cc(OC[C@@H](O)CO)ccc2C#Cc2ccc(OC[C@@H](O)CO)cc21]

Amine 23: TFA (0.5 ml) was added to a mixture of amide 22 (30 mg, 0.042 mmol), iPr₃SiH (0.1 ml), water (0.1 ml) and CH₂Cl₂ (1 ml). The reaction was stirred at room temperature for 3 h, diluted with CH₂Cl₂ and extracted with water. The combined aqueous phases were lyophilized to give crude deprotectd amine.

The residue was dissolved in MeOH (3 ml) and irradiated (360 nm) for 45 min. All volatiles were removed under reduced pressure and the resultant residue was purified by reversed phase HPLC chromatography (Chromolith RP18e, C₁₈, 15-31% MeCN in H₂O, 0.1% TFA, over 30 min) to give amine 23 (18.7 mg, 0.027 mmol, 64% over 2 steps) a white solid.

¹H NMR (400 MHZ, d₄-MeOH) δ=7.32 (d, J=8.66, 1H), 7.23 (d, J=2.51 Hz, 2H), 7.14 (d, J=8.53 Hz, 1H), 7.06 (dd, J=8.60, 2.57 Hz, 1H), 6.92 (dd, J=8.47, 2.32 Hz, 1H), 5.04 (d, J=14.2 Hz, 1H), 4.06 (m, 6H), 3.87 (dd, J=17.1, 1.69 Hz, 1H), 3.76 (d, J=14.1 Hz, 1H), 3.68 (m, 5H), 3.51 (t, J=5.84 Hz, 2H), 3.07, m, 2H), 2.85 (m, 1H), 2.29 (m, 2H), 2.07 (m, 1H) ppm.

¹³C NMR (150 MHz, d₄-MeOH): δ=174.7, 173.0, 171.8, 159.2, 159.2, 158.5, 151.9, 149.6, 127.0, 125.9, 119.2, 119.2, 116.2, 115.5, 114.6, 114.4, 112.8, 106.3, 70.3, 70.2, 69.5, 69.2, 62.7, 62.6, 42.5, 39.7, 36.6, 30.5, 29.8 ppm.

MS (ESI): calcd. for C₂₉H₃₇N₄O₉⁺: 585.3 [M+H]⁺
found: 585.5 [M+H]⁺.

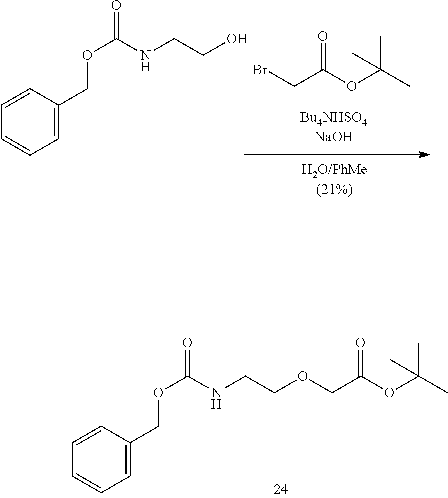

Ether 24: NaOH (30%, 42 ml) was added slowly to a mixture of N-Z-ethanolamine (4.00 g, 20.5 mmol), tert-butyl bromoacetate (6.06 ml, 41.0 mmol) and Bu₄NHSO₄ (2.96 g, 8.72 mmol) in PhMe (84 ml) and the reaction was allowed to stir at room temperature overnight. tert-Butyl bromoacetate (1.74 ml, 11.8 mmol) was added and the reaction was stirred at room temperature for 6 h. The layers were separated and the organic phase was washed with 5% AcOH and water, dried over MgSO₄ and concentrated under reduced pressure. Purification of the resulting residue by flash column chromatography (EtOAc:hexane=1:4) gave ether 24 (1.35 g, 4.36 mmol, 21%) as a colorless oil.

¹H NMR (400 MHZ, CDCl₃) δ=7.34 (m, 4H), 5.44 (brs, 1H), 5.10 (s, 2H), 3.95 (s, 2H), 3.61 (m, 2H), 3.41 (m, 2H), 1.46 (s, 9H) ppm.

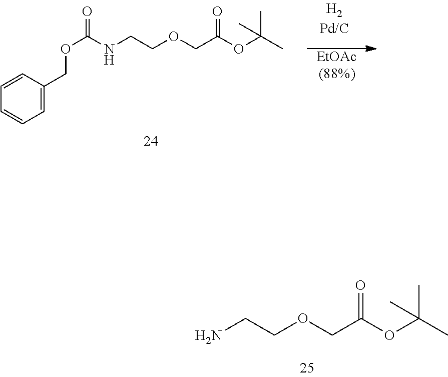 [SMILES: CC(C)(C)OC(=O)COCCNC(=O)OCc1ccccc1 ; CC(C)(C)OC(=O)COCCN]

Amine 25: Pd/C (710 mg) was added to a solution of ether 24 (1.35 g, 4.36 mmol) in EtOAc (15 ml) and the reaction vessel was set under a H₂-atmosphere. The reaction was stirred at room temperature for 3 h and then filtered over celite. Concentration of the solution under reduced pressure gave amine 25 (670 mg, 3.82 mmol, 88%) as a colorless oil.

¹H NMR (400 MHZ, CDCl₃) δ=3.99 (s, 2H), 3.57 (t, J=5.04 Hz, 2H), 2.91 (t, J=5.20 Hz, 2H), 1.87 (brs, 2H), 1.48 (s, 9H) ppm.

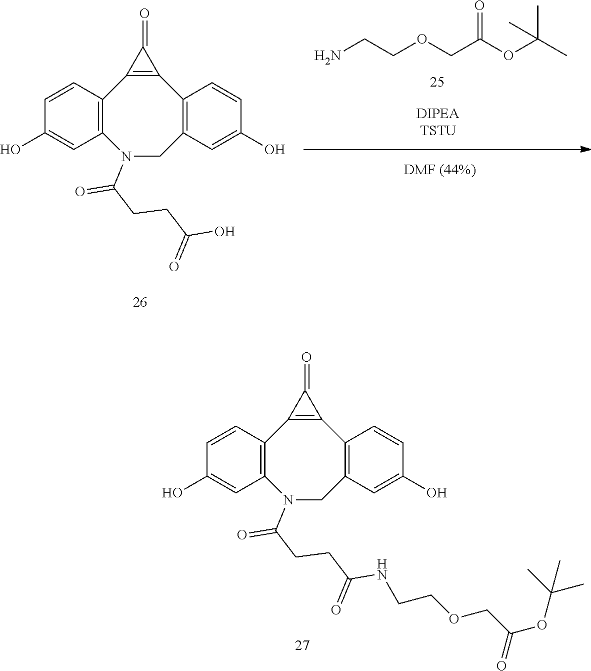 [SMILES: O=C(O)CCC(=O)N1Cc2cc(O)ccc2-c2c(c2=O)-c2ccc(O)cc21 ; CC(C)(C)OC(=O)COCCN ; CC(C)(C)OC(=O)COCCNC(=O)CCC(=O)N1Cc2cc(O)ccc2-c2c(c2=O)-c2ccc(O)cc21]

Amide 27: DIPEA (48 μL, 0,178 mmol) was added to a solution of carboxylic acid 26 (50 mg, 0.137 mmol) and TSTU (54 mg, 0.178 mmol) in DMF (2 ml) and the reaction was stirred at room temperature for 2 h. Amine 25 (31 mg, 0.178 mmol) was added and the reaction was stirred at room temperature for 4 h and concentrated under reduced pressure. Purification of the resulting residue by reversed phase HPLC chromatography (Chromolith RP18e, C₁₈, 35-51% MeCN in H₂O, 0.1% TFA, over 30 min) gave amide 27 (31.6 mg, 60 μmol, 44%) as a white solid.

¹H NMR (400 MHz, d₄-MeOH) δ=7.91 (d, J=8.71 Hz, 1H), 7.76 (d, J=8.31 Hz, 1H), 7.13 (d, J=2.37 Hz, 1H), 7.05 (d, J=1.98 Hz, 1H), 7.01 (dd, J=8.31, 2.37 Hz, 1H), 6.86 (dd, J=8.51, 2.18 Hz, 1H), 5.09 (d, J=14.6 Hz, 1H), 4.21 (d, J=14.3 Hz, 1H), 3.95 (s, 2H), 3.45 (m, 2H), 3.23 (m, 2H), 2.68 (m, 1H), 2.34 (m, 1H), 2.23 (m, 1H), 2.06 (m, 1H), 1.46 (s, 9H) ppm.

MS (ESI): calcd. for C₁₆H₂₄NO₅⁺: 523.2 [M+H]⁺
found: 523.4 [M+H]⁺.

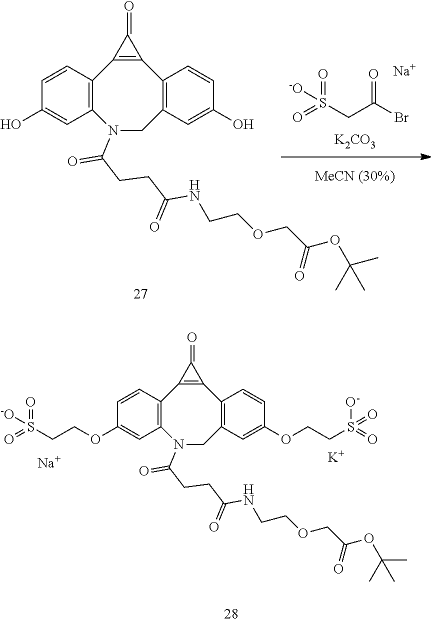

Sulfonic acid 28: A suspension of amide 27 (100 mg, 0.191 mmol), K₂CO₃ (132 mg, 0.955 mmol) and sodium 2-bromoethanesulfonate (202 mg, 0.955 mmol) in MeCN (2.0 ml) was stirred at 80° C. for 20 h. Purification of the reaction mixture by reversed phase HPLC chromatography (Chromolith RP18e, C₁₈, 22-38% MeCN in H₂O, 0.1% TFA, over 30 min) gave sulfonic acid 28 (46 mg, 0.058 mmol, 30%) as a white solid.

MS (ESI): calcd. for C₃₂H₃₉N₂O₁₄S₂⁺: 739.2 [M+H]⁺
found: 739.3 [M+H]⁺.

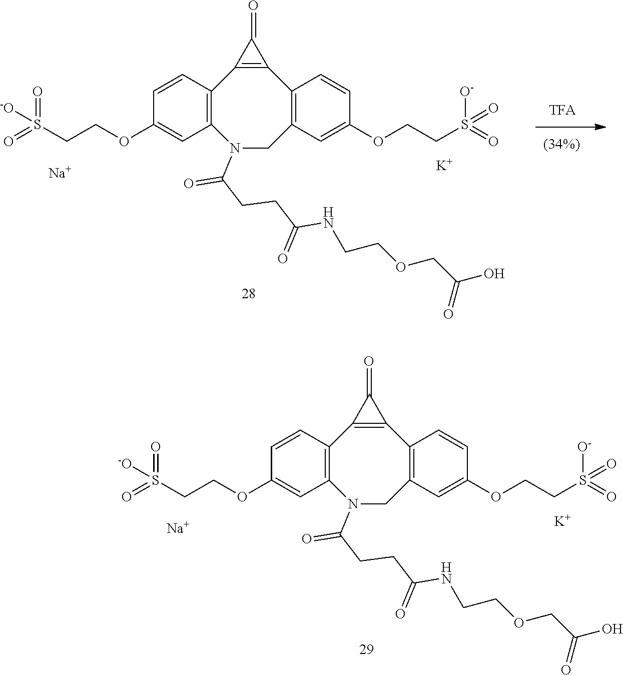

Carboxylic acid 29: : A solution of sulfonic acid 28 (45 mg, 0.061 mmol) in CH₂Cl₂ (2 ml), iPr₃SiH (0.2 ml), water (0.2 ml) and TFA (1 ml) was stirred at room temperature for 6 h, diluted with water and lyophilized. Purification of the resultant residue by reversed phase HPLC chromatography (Chromolith RP18e, C₁₈, C18, 16-32% MeCN in H₂O, 0.1% TFA, over 30 min) gave carboxylic acid 29 (15.2 mg, 0.021 mmol, 34%) as a white solid.

MS (ESI): calcd. for C₂₈H₃₁N₂O₁₄S₂⁺: 683.1 [M+H]⁺
found: 683.3 [M+H]⁺.

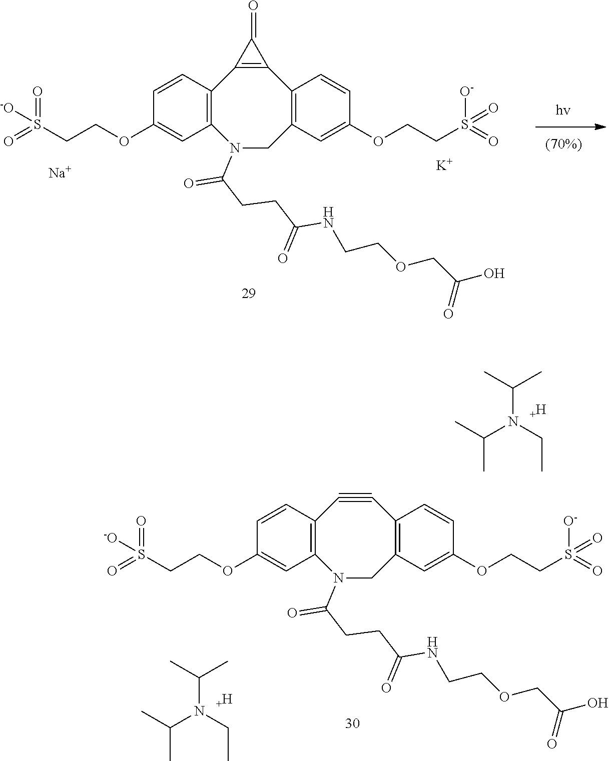

Alkyne 30: A solution of carboxylic acid 29 (17 mg, 0.023 mmol) in MeOH (2 ml) and DIPEA (0.10 ml) was irradiated (360 nm) for 0.5 h and concentrated under reduced pressure. Purification of the resultant residue by reversed phase HPLC chromatography (YMC-Triart C18, 18-30% MeCN in H₂O, 0.1% TFA, over 30 min) gave carboxylic acid 30 (16.7 mg, 0.018 mmol, 80%) as a white solid.

MS (ESI): calcd. for C₂₇H₃₁N₂O₁₃S₂⁺: 655.1 [M+H]⁺
found: 655.3 [M+H]⁺.

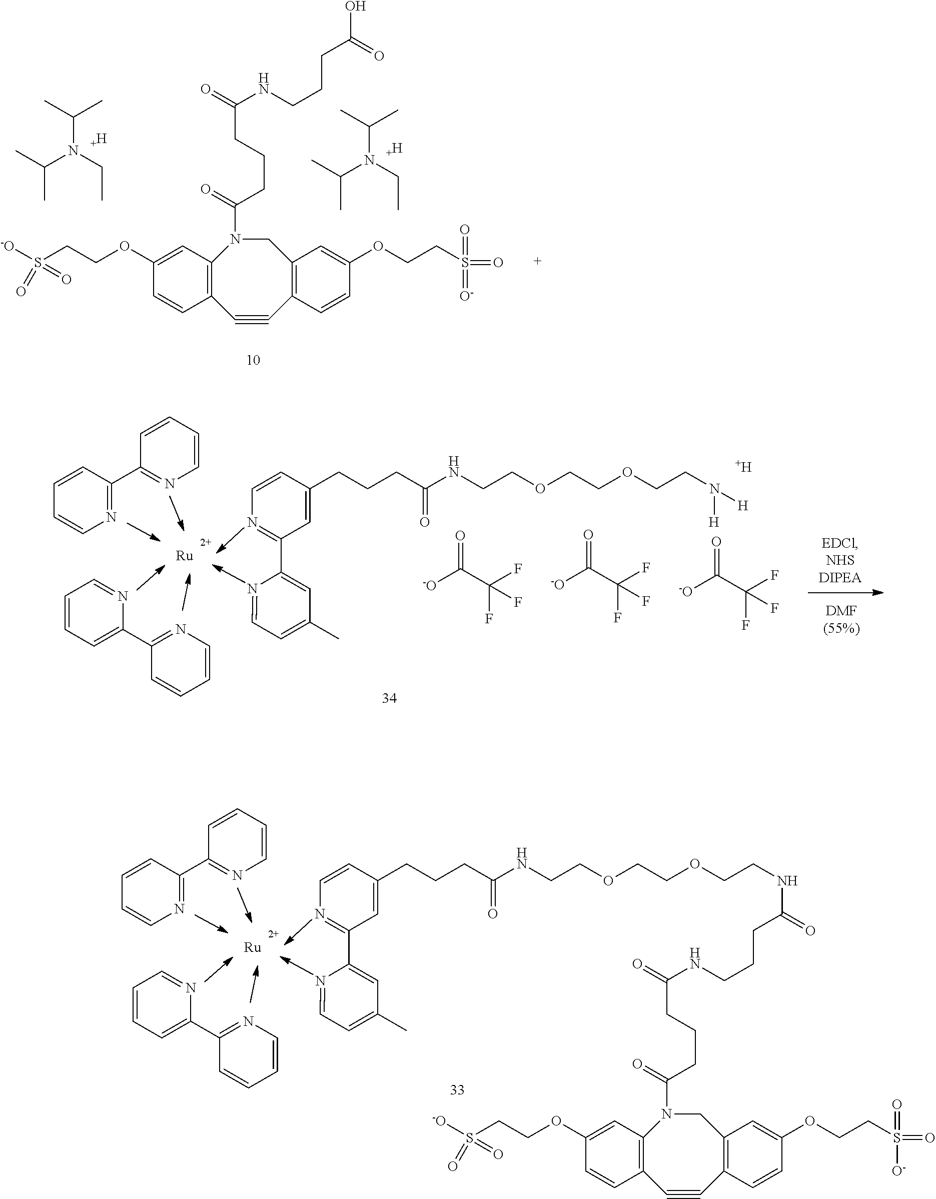

Ruthenium complex 33: DIPEA (28 μL, 0.158 mmol), NHS (5.0 mg, 43.5 μmol) and EDC HCl (15.2 mg, 79.0 μmol) were sequentially added to a solution of 10 (36 mg, 40 μmol) in DMF (1.0 ml) and the reaction was allowed to stir at room temperature for 2 h. Amine 34 (45 mg, 40 μmol) was added and the reaction was stirred at room temperature overnight. The solvent was removed under reduced pressure and the resulting residue was purified by HPLC-chromatography (C18, 2-90% MeCN in H₂O, 0.05% TFA, over 90 min) to give ruthenium complex 33 (31 mg, 21.6 mmol, 55%) as a red solid.

MS (ESI): calcd. for C₆₉H₇₄N₁₀O₁₄S₂Ru⁺: 717.20 [M+2H]²⁺/2
found: 717.27 [M+2H]²⁺/2.

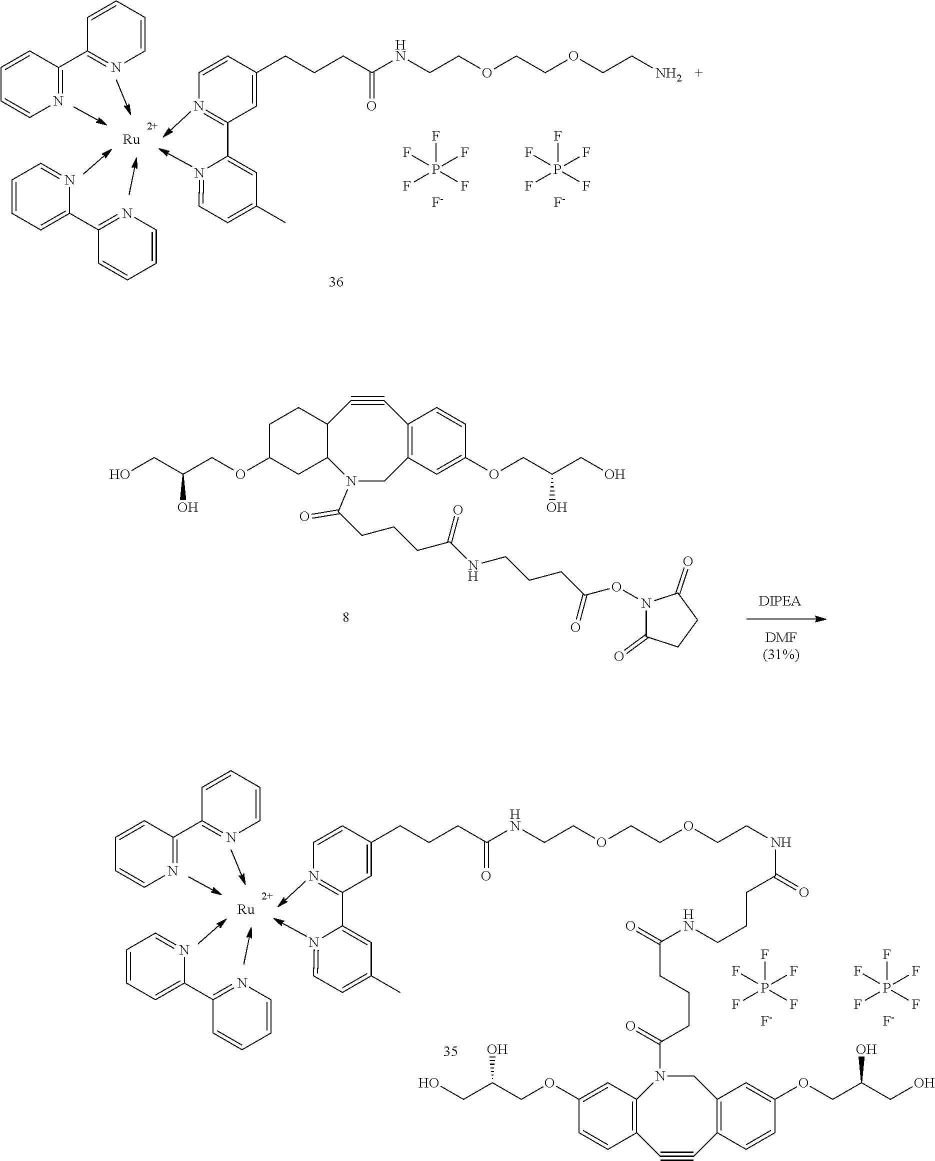

Ruthenium complex 35: A solution of amide 36 (7.7 mg, 7.1 mmol), NHS-ester 8 (7.2 mg, 10.6 mmol), and DIPEA (2.5 μL. 14.4 μmol) in DMF (1.0 ml) was stirred at room temperature for 1 d. The solvent was removed under reduced pressure and the resulting residue was purified by HPLC-chromatography (C18, 0-100% MeCN in H₂O, 0.1% TFA, over 80 min) to give ruthenium complex 35 (3.7 mg, 2.2 mmol, 31%) as a red solid.

MS (ESI): calcd. for C₇₁H₈₀N₁₀O₁₂Ru⁺: 683.26 [M+2H]²⁺/2
found: 683.39 [M+2H]²⁺/2.

### Solubility Experiments

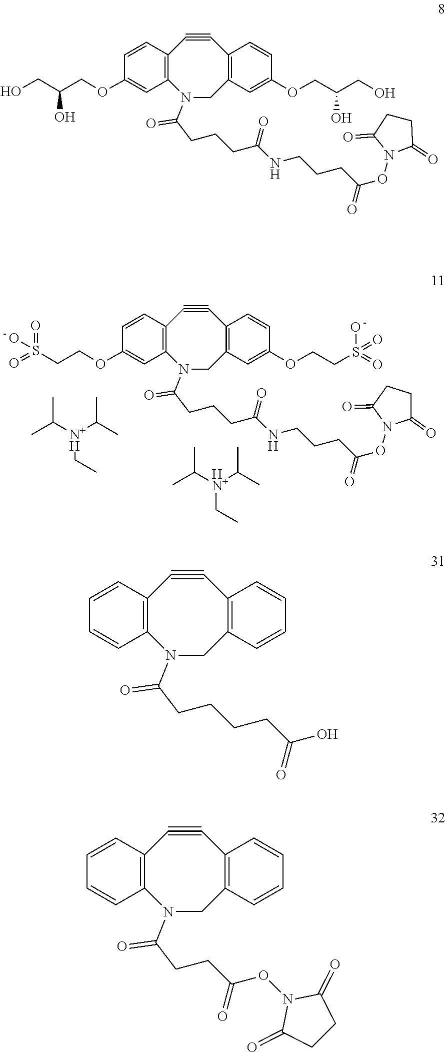

Inventive compound 8 (16.9 mg, 0.0248 mmol), inventive compound 11 (10.4 mg, 0.0139 mmol), standard compound 31 (5.0 mg, 0.015 mmol) and standard compound 32 (4.2 mg, 0.0104 mmol) were each successively mixed with water to reach a theoretical concentration of 515 mM, 16.5 mM, 12.4 mM, 6 mM and 0.6 mM. After each addition the mixture was sonicated for 10 s. If solid compound was still visible, this was considered as not dissolved. If no solid compound was visible, this was considered as dissolved. The results of the visual inspection are listed in Table 1, wherein “yes” or “no” indicate whether the compound was considered dissolved based on visual inspection.

TABLE 1
Comparison of the solubility of standard compounds 31
and 32 and compounds 8 and 11 according to invention

| Compound | 515 mM | 16.5 mM | 12.4 mM | 6.0 mM | 0.6 mM |
|---|---|---|---|---|---|
| 11 | yes | yes | yes | yes | yes |
| 8 | no | no | yes | yes | yes |
| 32 | no | no | no | no | no |
| 31 | no | no | no | no | no |

The inventive compounds have a significant higher hydrophilie as shown by their better solubility in water. It is apparent that from the inventive compounds, those carrying SO₃⁻ groups are even more hydrophilic than the inventive compounds having hydroxyl groups.

### Hydrophobicity Indicated by HPLC

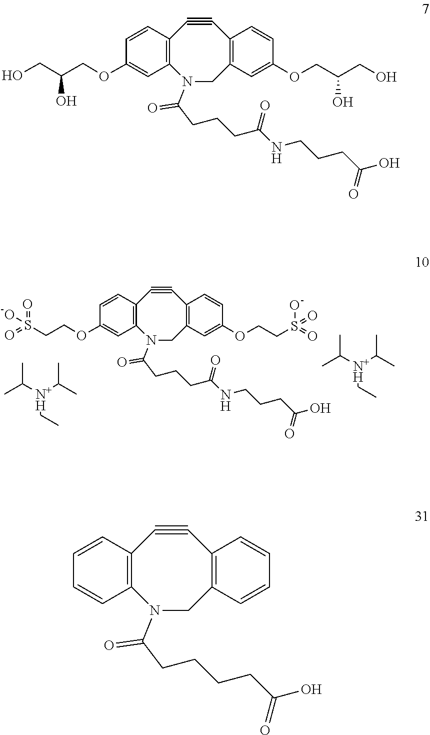

HPLC-chromatogram on a reverse phase (RP HPLC, YMC-Triart C18, 0-100% MeCN in H₂O, 5 0.1% TFA in 25 min) of inventive compounds 7 and 10 as well as standard compound 31 were taken, the respective retention times are listed in Table 2. A later retention corresponded to better interactions with the hydrophobic stationary phase and the compound could therefore be considered as more hydrophobic. The other way round, an earlier retention corresponded to higher hydrophilie.

TABLE 2
Retention time on YMC-Triart C₁₈, 0-100%
MeCN in H₂O, 0.1% TFA in 25 min

| Compound | Retention Time |
|---|---|
| 31 | 15.18 min |
| 7 | 11.58 min |
| 10 | 9.45 min |

It could be seen that the inventive compounds are more hydrophilic, wherein it was apparent that the inventive compounds having SO3 groups were even more hydrophilic than the inventive compounds having hydroxyl groups.

### Antibody Conjugation

Modified target molecule comprising the reaction product of a conjugate of formula (II) and a target molecule, here a MAB<Tn-T>chim-5D8-IgG antibody (anti Troponin T monoclonal IgG antibody manufactured by Roche in Penzberg/Germany, Roche material no. 05074991001; the monoclonal antibody 5D8 is known to the art, e.g. from Jaffe A. S. et al. Journal of the American College of Cardiology 58 (2011) 1819-1824), comprising a 1,3-dipole group, here an azide group, were prepared. These target molecule conjugates were also abbreviated in the following as “conjugates”.

The MAB<Tn-T>chim-5D8-IgG antibody was treated with an increasing excess (5, 10, 15 and 20-fold) of NHS-PEG5-DBCO (for the reference compound; entry 1-4) or NHS-PEG4-azide (for the inventive compounds; entry 5-12). The unconjugated excess labels were removed by dialysis. These conjugates were further treated with an 3-fold excess (compared to the previously used NHS ester) of BPRu-(O20c)₃-azide (entry 1-4) or 33 (entry 5-8) or 35 (entry 9-12) respectively. After the reaction, the unconjugated excess labels were separated by size exclusion chromatography by Superdex™ 200 Increase 10/300 GL using 5% DMSO in

Phosphate buffer 50 mM K⁺ phosphate, 150 mM KCl pH 7.4. Antibodies treated with 33 achieved higher incorporation of the label compared to the reference compound (table 3).

TABLE 3
Conjugation of label to the antibody

| | | | DBCO or | | | | | | | |
|---|---|---|---|---|---|---|---|---|---|---|
| | | | Azide/IgG MS | Ab:Ru | Ratio | incorporation | conc | Vol. | Amount | Product |
| No. | Antibody | Label | incorporation | 1: | 455/280 | Label/IgG | mg/ml | ml | mg | Yield % |
| 1 | TnT-IgG-PEG5- | BPRu-(O2Oc)3-Azide | — | 10 | 0.091 | 4 | 0.346 | 0.19 | 0.066 | 13 |
| 2 | DBCO | (R144) | — | 21 | 0.125 | 5.8 | 0.437 | 0.28 | 0.122 | 24 |
| 3 | | | — | 33 | 0.138 | 8.2 | 0.256 | 0.382 | 0.098 | 20 |
| 4 | | | — | 45 | 0.145 | 11.2 | 0.142 | 0.337 | 0.048 | 10 |
| 5 | TnT-IgG-PEG4- | BPRu-PEG3-sulfoDBCO | 3.4 | 10 | 0.116 | 4.3 | 0.957 | 0.333 | 0.319 | 64 |
| 6 | Azide | (R155) | 7.5 | 21 | 0.146 | 8.2 | 0.626 | 0.444 | 0.278 | 56 |
| 7 | | | 11 | 33 | 0.159 | 12.1 | 0.44 | 0.489 | 0.215 | 43 |
| 8 | | | 15 | 45 | 0.166 | 15.4 | 0.261 | 0.534 | 0.139 | 28 |
| 9 | TnT-IgG-PEG4- | BPRu-PEG3- | 3.4 | 10 | 0.103 | 3.5 | 0.697 | 0.278 | 0.194 | 39 |
| 10 | Azide | dihydroxyDBCO (R156) | 7.5 | 21 | 0.129 | 5.9 | 0.401 | 0.407 | 0.163 | 33 |
| 11 | | | 11 | 33 | 0.140 | 9.8 | 0.201 | 0.435 | 0.087 | 17 |
| 12 | | | 15 | 45 | 0.146 | 12.3 | 0.089 | 0.327 | 0.029 | 6 |

All conjugates were evaluated in a model sandwich immunoassay (Elecsys Troponin T hs, Roche: 05092744 190) containing streptavidin-coated beads (Roche: 05092744) measured on an Elecsys e170 modular using an ECL-signal as the readout. Measurements without analyte in buffer (Diluent Universal, Roche: 11732277 122) showed that 33 was able to reduce the background 71-281 fold and 35 2-8 fold. Using serum (Diluent MultiAssay, Roche: 03609987 190), the background-signal reduction increased to 56-1065 fold for 33 and 9-62 fold for 35 (table 4).

The reduction of the background signal for 33 and 35 conjugates in comparison to that of reference DBCO conjugates can be attributed to hydrophilization (due to sulfonation or hydroxylation) of the otherwise hydrophobic moieties.

Measurements of Calibrator 2 (id. 05092752 190) lead to a signal which was in or slightly above the background when the reference conjugate was used (see Cal2/MA above). Using 33 and 35 the ratio of signal to background was increased up to 374 and 334 respectively (see Cal2/MA above).

Stability tests revealed a higher recovery of the ECL-signal for conjugates with 33 and 35 compared to the reference compound after storage for 8 days at 35° C.

TABLE 4
ECl-measurements of the antibody conjugates

| | | | | | | | | | | | Day 8 |
|---|---|---|---|---|---|---|---|---|---|---|---|
| | | | | | | | | | | | Cal2 |
| | | | | | Day 0 | | | | | | recovery |
| | | | | | Uni- | MA- | TNT- | TNT- | | | after 8 |
| | | | | Incorporation | Dil- | Dil- | Cal1- | Cal2- | Cal1/ | Cal2/ | days at |
| No. | Code | Antibody | Label | Label/IgG | PW | PW | PW | PW | MA | MA | 35° C. |
| 1 | R144-5 | TnT-PEG5-DBCO | BPRu-(O2Oc)3- | 4 | 102624 | 45512 | 15551 | 446878 | 0 | 10 | 89 |
| | | | Azide | | | | | | | | |
| 2 | R144-10 | TnT-PEG5-DBCO | BPRu-(O2Oc)3- | 5.8 | 1502759 | 915223 | 550613 | 1274193 | 1 | 1 | 89 |
| | | | Azide | | | | | | | | |
| 3 | R144-15 | TnT-PEG5-DBCO | BPRu-(O2Oc)3- | 8.2 | 4226361 | 2991802 | 2145384 | 3001804 | 1 | 1 | 87 |
| | | | Azide | | | | | | | | |
| 4 | R144-20 | TnT-PEG5-DBCO | BPRu-(O2Oc)3- | 11.2 | 5936047 | 4418477 | 3366187 | 4255854 | 1 | 1 | 87 |
| | | | Azide | | | | | | | | |
| 5 | R155-5 | TnT-PEG4-Azide | BPRu-PEG3- | 4.3 | 1409 | 810 | 1072 | 268391 | 1 | 331 | 94 |
| | | | sulfoDBCO | | | | | | | | |
| 6 | R155-10 | TnT-PEG4-Azide | BPRu-PEG3- | 8.2 | 5356 | 1184 | 2160 | 442854 | 2 | 374 | 93 |
| | | | sulfoDBCO | | | | | | | | |
| 7 | R155-15 | TnT-PEG4-Azide | BPRu-PEG3- | 12.1 | 30400 | 2808 | 8120 | 553236 | 3 | 197 | 93 |
| | | | sulfoDBCO | | | | | | | | |
| 8 | R155-20 | TnT-PEG4-Azide | BPRu-PEG3- | 15.4 | 83749 | 6059 | 23996 | 638850 | 4 | 105 | 92 |
| | | | sulfoDBCO | | | | | | | | |
| 9 | R156-5 | TnT-PEG4-Azide | BPRu-PEG3- | 3.5 | 13294 | 953 | 2076 | 318077 | 2 | 334 | 92 |
| | | | dihydroxyDBCO | | | | | | | | |
| 10 | R156-10 | TnT-PEG4-Azide | BPRu-PEG3- | 5.9 | 377650 | 14767 | 42735 | 810926 | 3 | 55 | 93 |
| | | | dihydroxyDBCO | | | | | | | | |
| 11 | R156-15 | TnT-PEG4-Azide | BPRu-PEG3- | 9.8 | 1613162 | 124105 | 275756 | 1359021 | 2 | 11 | 92 |
| | | | dihydroxyDBCO | | | | | | | | |
| 12 | R156-20 | TnT-PEG4-Azide | BPRu-PEG3- | 12.3 | 3342026 | 475372 | 780624 | 2078544 | 2 | 4 | 90 |
| | | | dihydroxyDBCO | | | | | | | | |

### CITED LITERATURE

EP 3 004 062 B1
WO 2014/189370 A1
Debets et al., Chem. Commun. 2010, 46, 97-99
Org. Biomol. Chem., 2014, 12, 5031-5037
U.S. Pat. No. 8,912,322 B2
WO 2003/002974 A2
WO 2012/107419 A1
WO 2012/107420 A1
WO 2014/019709 A2
WO 2014/019708 A1
WO 2017/153574 A1
P. H. Hagedorn et al., Drug Discovery Today 2018, 23(1), 101-114
WO 2019/243391 A1
WO 2020/245377 A1
Abbas et al., Cellular and Mol. Immunology, 4th ed., W.B. Saunders, Co. (2000)
Plueckthun, In: The Pharmacology of Monoclonal Antibodies, Vol. 113, Rosenburg and Moore (eds.), Springer-Verlag, New York (1994) pp. 269-315
EP 0 404 097 A1
WO 1993/01161 A1
Hudson, P. J. et al., Nat. Med. 9 (2003) 129-134
Holliger, P. et al., PNAS USA 90 (1993) 6444-6448

1. An azadibenzocyclooctyne derivative according to formula (I) or a salt thereof, 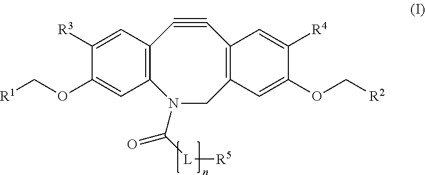
wherein
R¹, R² are the same and both a —[(CH₂)_{aCR}^{x}R^{y]}_{b}R^{z} group, wherein a is either zero or an integer from the range of from 1 to 4, b is either zero or 1,
R^{x}, R^{y}, R^{z} are selected from the group consisting of hydrogen atom, C1 to C3 alkyl group and (CH₂)_{c}SO₃⁻ group, with c being either zero or an integer from the range of from 1 to 4, wherein at least one of R^{x}, R^{y}, R^{z} is a (CH₂)_{c}SO₃⁻ group with the condition:
-if R^{z} is a (CH₂)_{c}SO₃⁻ group with c being zero, then R^{x}, R^{y} are not both a (CH₂)_{c}SO₃⁻ group wherein c is zero, or
if a is zero, then R^{x} and R^{y} are not both a (CH₂)_{c}SO₃⁻ group wherein c is zero;
R³, R⁴ are independently selected from the group consisting of hydrogen atom, C1-C3-alkyl group, halogen atom and -O-C1-C3-alkyl group; and
R⁵ is selected from the group consisting of carboxyl group, activated carboxyl group and —NHR^{5a} group, wherein R^{5a} is a hydrogen atom or a C1-C5 alkyl group;
L comprises a chain of covalently bonded atoms forming a backbone and having a length in the range of from 1 to 100 atoms (linker); and
n is either zero or 1 if R⁵ is a carboxyl group or an activated carboxyl group or n is 1 if R⁵ is a —NHR^{5a} group.

2. The azadibenzocyclooctyne derivative or salt thereof of claim 1, wherein R³, R⁴ are independently a hydrogen atom or a methyl group, preferably R³, R⁴ are identical and are each a hydrogen atom.

3. The azadibenzocyclooctyne derivative or salt thereof of claim 1 having formula (Ia) or (Ib): 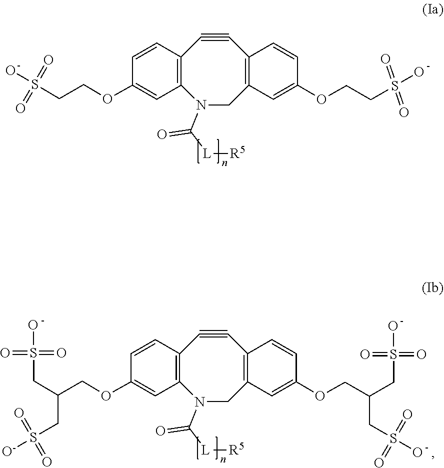
wherein L, n and R⁵ are as defined in claim 1.

4. The azadibenzocyclooctyne derivative or salt thereof of claim 1 having formula (Ia-1) or (Ib-1): 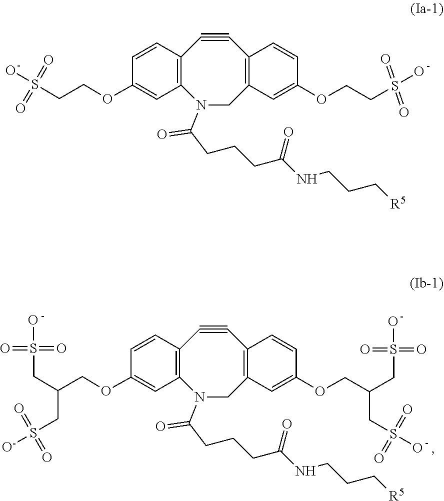
wherein R⁵ is as defined in claim 1.

5. A conjugate of formula (II) 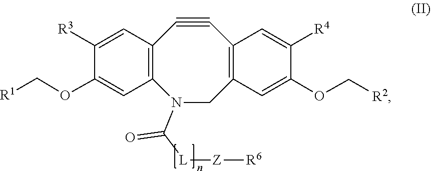
wherein L, R¹, R², R³, R⁴ and n are as defined in claim 1 for the azadibenzocyclooctyne derivative of formula (I) or salt thereof; and wherein
R⁶ is selected from the group consisting of fluorophore, hapten, tyramine, polyethylene glycol chain, polypropylene glycol chain, mixed polyethylene/polypropylene glycol chain, metal complex, radioactive isotope, active pharmaceutical ingredient, carbohydrate, solid phase, lipid, amino acid, oligopeptide, polypeptide, nucleotide, oligonucleotide and polynucleotide; and is preferably a metal complex; and
Z is selected from the group consisting of —C(═O)—O—, —C(═O)—NR⁷—, and —NR⁷—C(═Y)—, wherein R⁷ is a hydrogen atom or a C1-C5 alkyl group and Y is an oxygen atom or a sulphur atom, preferably an oxygen atom.

6. A conjugate of formula (II) according to claim 5, wherein R⁶ is selected from the group consisting of fluorophore, fluorescence quencher, dye, hapten, tyramine, metal complex, radioactive isotope, active pharmaceutical ingredient (drug), carbohydrate, solid phase, lipid, amino acid, oligopeptide, polypeptide, nucleotide, oligonucleotide and polynucleotide; and is preferably a metal complex, wherein a further linker is present or absent between Z and R⁶, which is preferably selected from the group consisting of alkyl chain, polyethylene e glycol chain, polypropylene glycol chain and mixed polyethylene/polypropylene glycol chain.

7. The conjugate of claim 5, having formula (IIa) or (IIb): 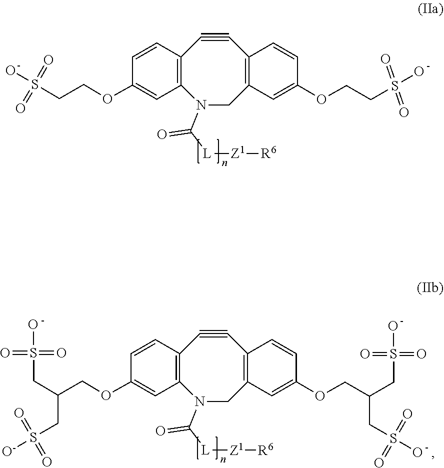
wherein L, n and R⁶ are as defined in claim 5 and Z¹ is a —C(═O)—O-group or a —C(═O)—NH-group.

8. The conjugate of claim 5, having formula (IIa-1), (IIa-2), (IIb-1) or (IIb-2): 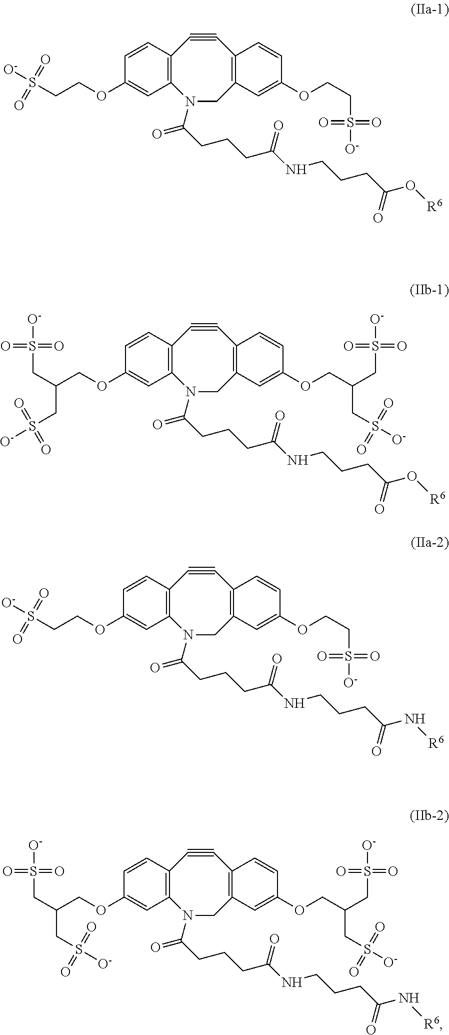
wherein R⁶ in (IIa-1), (IIa-2), (IIb-1) or (IIb-2) is as defined in claim 5.

9. A method for the modification of a target molecule, wherein a conjugate according to claim 5 is reacted with a target molecule comprising a 1,3-dipole group or a 1,3-(hetero)diene group.

10. The method according to claim 9, wherein the target molecule is selected from the group consisting of fluorophore, fluorescence quencher, dye, hapten, tyramine, polyethylene glycol chain, polypropylene glycol chain, mixed polyethylene/polypropylene glycol chain, metal complex, radioactive isotope, active pharmaceutical ingredient, carbohydrate, solid phase, lipid, amino acid, oligopeptide, polypeptide, nucleotide, oligonucleotide, and polynucleotide; and is preferably a polypeptide, more preferably an antibody, more preferably a modified antibody having a 1,3-dipole group, more preferably a modified antibody having an azide group.

11. (canceled)

12. A modified target molecule comprising the reaction product of a conjugate according to claim 5 and a target molecule comprising a 1,3-dipole group or a 1,3-(hetero)diene group.

13. A kit comprising a modified target molecule according to claim 12 as detector reagent and a suitable capture reagent.
